(19) United States
(12) Patent Application Publication
(10) Pub. No.: US 2009/0226494 A1
(43) Pub. Date: Sep. 10, 2009

(54) PATHOGEN - CONTROLLING PRODUCTS

(76) Inventor: Stephen Spaulding Hickok, London (GB)

Correspondence Address: Workman Nydegger;1000 Eagle Gate Tower, 60 East South Temple, Salt Lake City, UT, 84111, US

(21) Appl. No.: 12/094,115

(22) PCT Filed: Nov. 17, 2006
(86) PCT No.: PCT/GB06/04285

§ 371 Date: Nov. 18, 2008

(30) Foreign Application Priority Data
Nov. 17, 2005 (GB) 0523427.3
Sep. 7, 2006 (GB) 0617552.5

(51) Int. Cl. A01N 59/20; A61K 31/30; A01N 25/34

(52) U.S. Cl. 424/404; 424/630; 424/637; 514/499

(57) ABSTRACT

An antibacterial formulation which comprises: (a) at least one water soluble copper compound able to form copper ions upon dissolution in an aqueous medium; (b) at least one water soluble ammonium agent able to form ammonium ions upon dissolution in an aqueous medium; (c) at least one water soluble acid, and (d) an aqueous medium within which components (a), (b) and (c) are dissolved, said formulation having (e) an acidic pH and (f) an electrolytic potential in excess of 50 millivolts.

This invention is concerned with formulations and other products useful in the control of pathogenic disease and in combating the presence of pathogenic species likely or liable to cause infection. In the group of pathogenic organisms, bacteria, fungus and virus are classified. It is desirable to continue the pursuit for anti-infective agents and disinfectants capable of controlling pathogenic organisms in the free state (i.e. as may be present in the environment or surroundings) and in the infective state where pathogenic organism has invaded a host's body resulting in disease symptoms associated with the particular organism.

Conventionally, many diseased states attributed to pathogenic organisms are treated with antibiotics, typically drugs which have been discovered and commercialised for use in treating such infection. In the hospital ward, or clinic, theatre, surgery or similar environment, commercially available disinfectants are used as a preventative measure to control and in particular to kill or otherwise render harmless pathogens such as bacteria which may be present on surfaces such as floors, walls, basins, doors and the like. There are many widely available such disinfectants which tend to be halogenated/aromatic hydrocarbon based. Other chemical types are also known.

Commercially available disinfectants are also used in home and office environments, for example in homes and/or offices of healthcare workers. It is desirable to provide an infection control system for environments within or associated with hospitals.

However, there are current concerns with the emergence of antibiotic resistant pathogenic strains, for example: MRSA (methicillin-resistant Staphylococcus aureus); VRE (vancomycin-resistant Enterococcus); Helicobacter pylori resistant to clarithromycin, metronidazole to identify but a few. Antibiotic-resistant bacteria are problematical to treat with conventional antibiotics because of such acquired resistance. Accordingly it is desirable to provide alternative treatment and prevention regimes without reliance upon present or yet to be discovered antibiotic drugs.

There are also health concerns associated with aromatic halogenated disinfectants, and it is similarly desirable to develop alternative disinfectants and anti-infective agents not reliant upon halogenated aromatic components.

It has been suggested in our previous published application WO 01/15554 that a broad range of metallo-ion containing compositions may be beneficial in treating pathogenic organisms. We have now surprisingly found that a selection of compositions disclosed in our said earlier publication are useful in combating specific pathogenic organisms which are antibiotic resistant or otherwise difficult to treat or control, and/or which can be present in hospitals, surgeries, clinics and theatres, homes and the environment under a treatment regime without significant detriment or deleterious effect upon living human cells. We have also found such beneficial effects in compositions which are similar to but nonetheless different from those disclosed in our earlier said publication. We have also surprisingly found that a substrate can be impregnated with compositions described herein, to confer surprisingly effective and long lasting antibacterial properties. For example, we have found that a microfibre and/or ultramicro fibre cloth as currently commercially available for cleaning hospital surfaces can be impregnated with compositions described herein and used as a powerful wide spectrum antibacterial and/or antifungal disinfecting aid. We have also found that such microfibre cloth can be laundered, re-impregnated and reused many times, providing significant economic benefits.

We have also unexpectedly found that ionically modified copper-containing compositions described herein can be effective against multiple different pathogens simultaneously, and can provide protection against infection and re-infection with such multiple different pathogenic organisms.

The compositions described herein can be applied topically to a patient suffering an infection, for example topical application to the skin of a patient for preventing or treating MRSA and/or VRE.

We have further developed an infection control system based upon detection of the presence on a surface or within the atmospheric environment of at least one pathogenic organism by preferably micro fluidic assay, display or other presentation of the detection results, treatment of detected pathogenic species, by application to the surface or the atmospheric environment of one or more compositions of the type described herein, repetition of the detection step and repetition of the display step. Such a process of steps can lead to a substantive infection control system.

The compositions can be used or applied in a spray mist, fine mist or ‘fog’ for combating pathogenic species. In such application the composition acting as a disinfecting reagent is dissipated onto water droplets which are then applied as a fine spray or mist to cover exposed and/or hidden surfaces, and enter the cracks and crevices within building interiors. Once on the surface, the disinfecting properties of the complexed copper ion are effective and can remain effective for a considerable time. Various arrangements of spray can be used and the size of water droplets and concentration of applied composition varies. Surfactants can be included in these compositions for such purposes.

The invention also embraces detergent compositions which incorporate the present copper containing composition. In particular such detergents will become disinfecting and capable of controlling pathogenic species such as bacteria and drug resistant bacteria when used to launder clothing worn by healthcare workers or other people in contact with patients suffering an infection. Similarly such disinfecting detergents can be used to launder clothing and bed clothing of patients suffering an infection.

The compositions described below are conveniently prepared according to the general procedure outlined in our above referred to patent application save that the addition of acid can in some cases be limited to obtain electrolytic potential starting at a lower range, for example at least as high as 150 mVolts, but some embodiments being less than 350 mV. Where additional ingredients are present, e.g. surfactants to assist in surface cleansing anti infective products this is indicated in the table.

TABLE 1

| Embodiment | | Ammonium | | | Final | Electrolytic |
|---|---|---|---|---|---|---|
| No. | Compound/amount | Agent/amount | Acid/Amount | Additive(s) | pH | potential |
| 1 | Copper sulphate | Ammonium | Sulphuric | NIL | <2 | >150 |
| | 150 g | sulphate 75 g | 98% variable | | | |
| 2 | Copper sulphate | Ammonium | Sulphuric | NIL | <2 | >300 |
| | 150 g | sulphate 75 g | 98% variable | | | |
| 3 | Copper sulphate | Ammonium | Sulphuric | NIL | 1.5 | >150 |
| | 200 g | sulphate 75 g | 98% variable | | | |
| 4 | Copper sulphate | Ammonium | H₃PO₄ | NIL | 1-2 | >150 |
| | 150 g | phosphate 75 g | variable | | | |
| 5 | Copper sulphate | Ammonium | HCL conc | NIL | <2 | >150 |
| | 150 g | Chloride 75 g | variable | | | |
| 6 | Copper sulphate | Ammonium | H₂SO₄ conc | NIL | <2 | >150 |
| | 200 g | sulphate 75 g | Variable | | | |
| 7 | Copper sulphate | Ammonium | HCL conc | NIL | <2 | >300 |
| | 150 g | Chloride 75 g | variable | | | |
| 8 | Copper sulphate | Ammonium | H₂SO₄ conc | NIL | >2 | >150 |
| | 300 g | sulphate 82.5 g | Variable | | | |
| 9 | Copper sulphate | Ammonium | H₂SO₄ conc | Surfactant(s) | <2 | >150 |
| | 150 g | Sulphate 75 g | variable | | | |

In other embodiments a lower concentration of copper is desirable, for example 80 to 140 g such as 90 to 130 g or of the order 100 to 120 g of copper sulphate, given for same quantities for other components. This can be useful for topical applications and against H. pylori infection.

In the above Table, the compositions present as copper-containing aqueous solutions in which the copper is present as dissolved metallo ion, in the presence of and potentially combined with aqueous ammonium ions from the dissolved ammonium agent and the compositions exhibiting demonstrable electrolytic potentials of at least 150 mV although in some preferred embodiments greater than 300 mV, such as at least 350 mV. We have surprisingly found that the aforesaid compositions can be highly effective against difficult to treat bacterial strains such as of persistent strains of E. coli with simultaneous lack of cytotoxicity to at least two different human cell cultures for example HT-29 and U-937 human cells, when applied at a concentration of less than 100 ppm, e.g. 50 ppm, to cultures of these E. coli cells. However, concentrations as high as 1000 ppm of equivalent copper are contemplated in some embodiments.

It is preferred that the equivalent concentration of copper in the compositions is of the order 10 to 50 g/Litre, preferably 20 to 40 g/Litre, more preferably 25 to 35 g/Litre, the solvent phase being distilled (in contrast to deionised) water.

It is preferred for the target pathogenic organisms be treated with composition containing in the range of 0.01 to 100 ppm of equivalent copper, at ambient temperature and for a duration of 1 minute to 12 hours, or 1 minute to 6 hours or 0.25 up to 3.0 hours. However, in the case of spray/fogging treatments, the application time can be much shorter as the sprays can be in short bursts.

The present copper compositions can be used at, e.g. 0.5 to 500 ppm of equivalent copper against Helicobacter pylori (H. pylori) and especially against drug resistant Helicobacter pylori both of which are major causes of gastric/peptic ulcers. The resistant strains especially treatable by the present copper compositions are clarithromycin resistant H. pylori, metronidazole resistant H. pylori and (although rare) amoxicillin resistant H. pylori.

The present copper compositions can be formulated into topical formulations such as creams, gels, and spray solutions which can be for application to the skin and mucosal surfaces, impregnated dressings and irrigation solutions.

The present copper compositions can be used to impregnate an absorbent substrate useful for cleaning surfaces, so as to disinfect such surfaces. The preferred substrate is termed microfibre and/or ultramicro fibre (UMF) cloth available from Johnson Diversity, Inc. As foreshadowed above, such impregnated microfibre cloths can be laundered and reused many times. Impregnated, such cloths provide a ready means of controlling bacterial growth and/or development, e.g. inhibiting bacterial growth and/or development, e.g. inhibiting bacterial growth and/or replication or at least inhibiting bacterial activity of such bacteria. Whilst the present invention in its broad scope is wide enough to embrace the combination of microfibre substrate impregnated with any antimicrobial agent, the invention also includes the specific embodiment of such microfibre substrate impregnated with a copper composition derived from the above table, or otherwise in accordance with copper-containing compositions as fall within the scope of this invention.

An advantage of incorporating the present copper based metallo-ion biocides within the substrate such as the microfibre or ultramicrofibre cloth is that it can prevent cross contamination of surfaces which is a real danger without it.

In particular such impregnated microfibre cloth can be used to disinfect surfaces (e.g. as in hospitals, surgeries, clinics, theatres) against the difficult to treat nosocomial hospital infections MRSA (wild strain), ACCB (wild strain), VRE (wild strain), C. diff (spore suspension), LPn (Legionella) as subsequently defined herein and Salmonella.

The present compositions and substrates impregnated therewith can provide a very substantial and significant inhibition of bacterial activity, i.e. are capable of interfering with and thereby controlling the growth, development and/or replication of such nosocomial pathogenic bacteria hitherto difficult to treat with conventional antibiotic and/or conventional disinfectant regimes. Such inhibition of bacterial pathogenic activity can be surprisingly accomplished without significant concomitant cytotoxicity to prevalent surrounding human cells.

The invention is defined herein in the accompanying claims.

In order that the invention may be illustrated, more easily appreciated and readily carried into effect by those skilled in the art, embodiments thereof will now be presented by way of non-limiting example only and described with reference to the accompanying drawings, wherein:

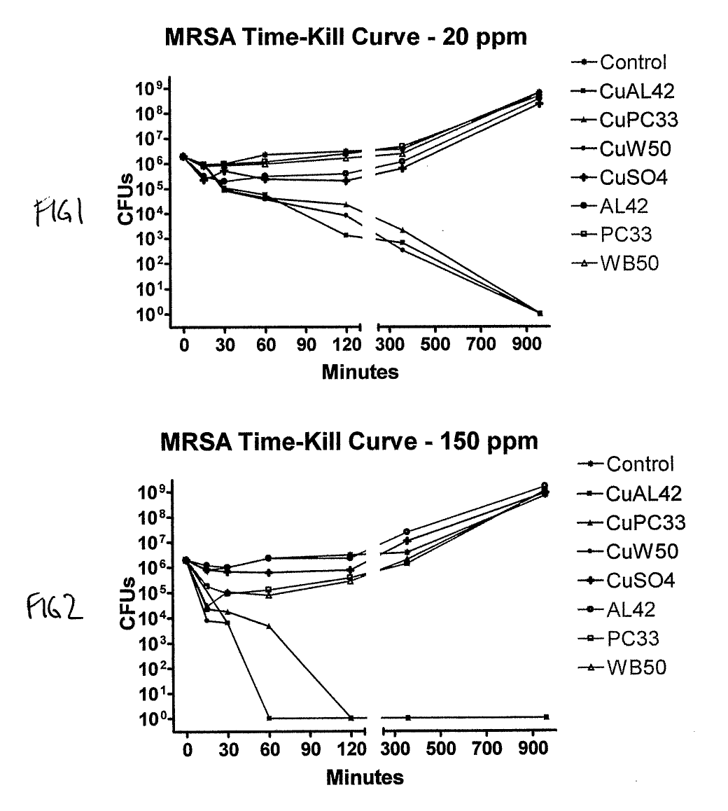

FIG. 1 is an MRSA time-kill curve at 20 ppm equivalent copper for the compositions, the copper salt alone and the remaining components of the composition (colloquially referred to herein as the ‘binder’) for comparison,

FIG. 2 is a similar MRSA time-kill curve to FIG. 1, but at 150 ppm of equivalent copper,

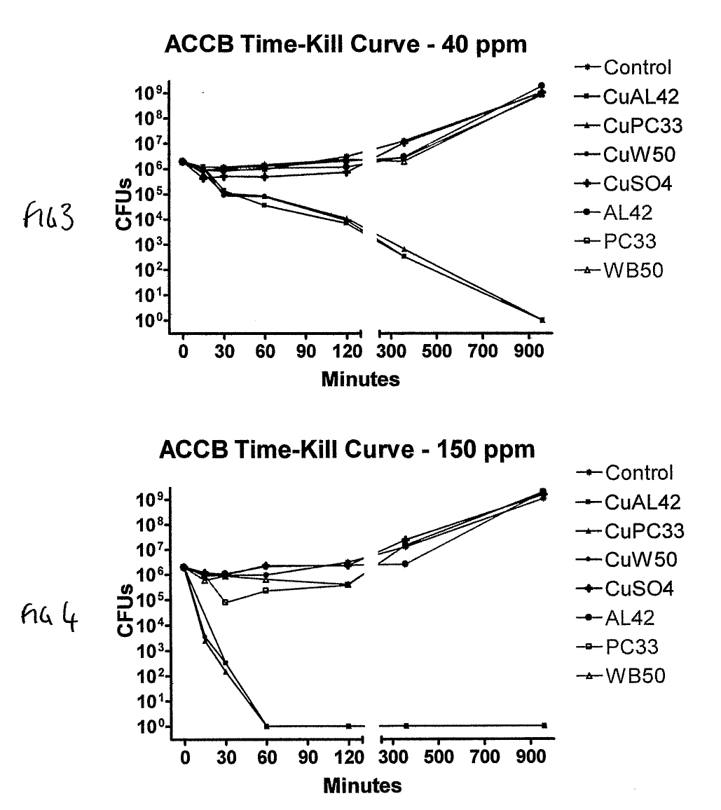

FIG. 3 is a similar ACCB time-kill curve to FIG. 1, at 40 ppm,

FIG. 4 is a similar ACCB time-kill curve to FIG. 3, at 150 ppm,

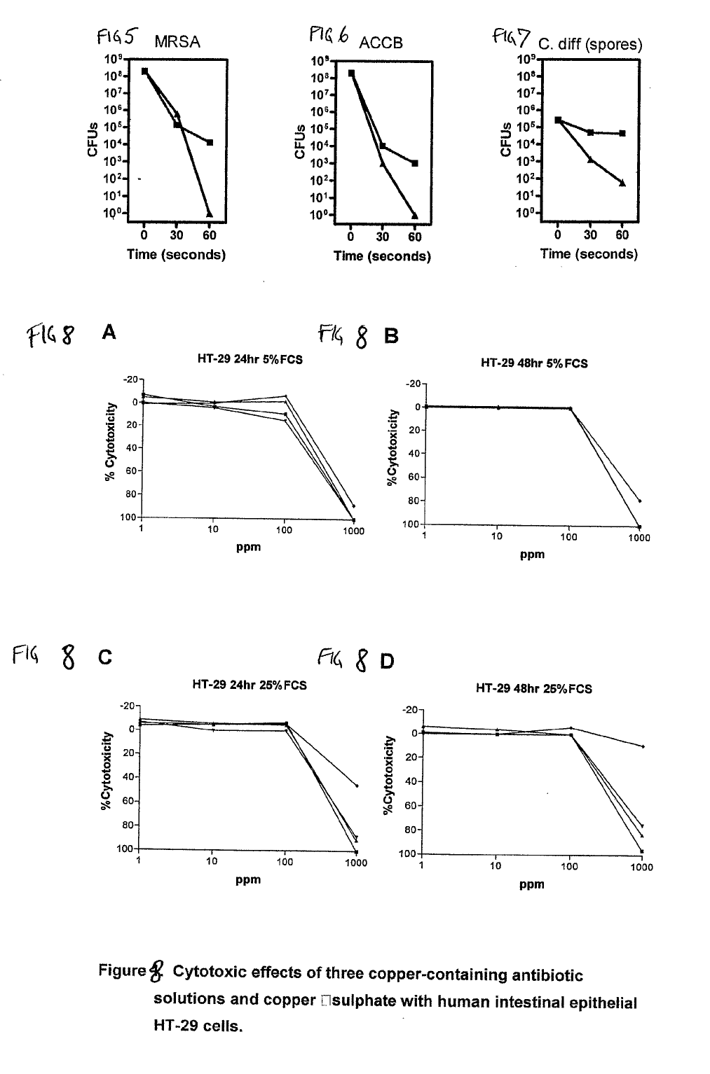

FIG. 5 demonstrates the antibacterial effects of the formulated X-gel aqueous medium containing CuAL42 [▴] and Purell™ [▪] hand gels on the survival of MRSA bacteria using the standard EN 12054 protocol,

FIG. 6 is a view similar to FIG. 5, but demonstrating effects using the same formulations upon the survival of ACCB,

FIG. 7 is a view similar to FIGS. 5 and 6, but demonstrating effects using the same formulations upon the survival of C. diff (spores),

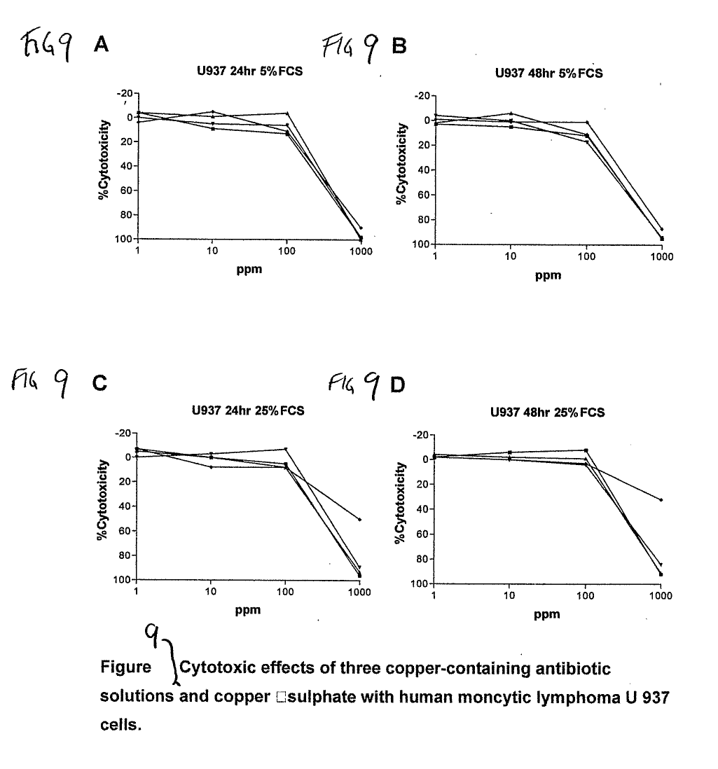

FIGS. 8A to 8D are graphs representing the cytotoxic effects of the three copper formulations and copper sulphate alone [□] upon human intestinal epithelial HT-29 cells,

FIGS. 9A to 9D are graphs similar to FIGS. 8A to 8D, but showing the cytotxic effects of the three copper formulations compared with copper sulphate alone [□] upon human monocytic lymphoma U937 cells,

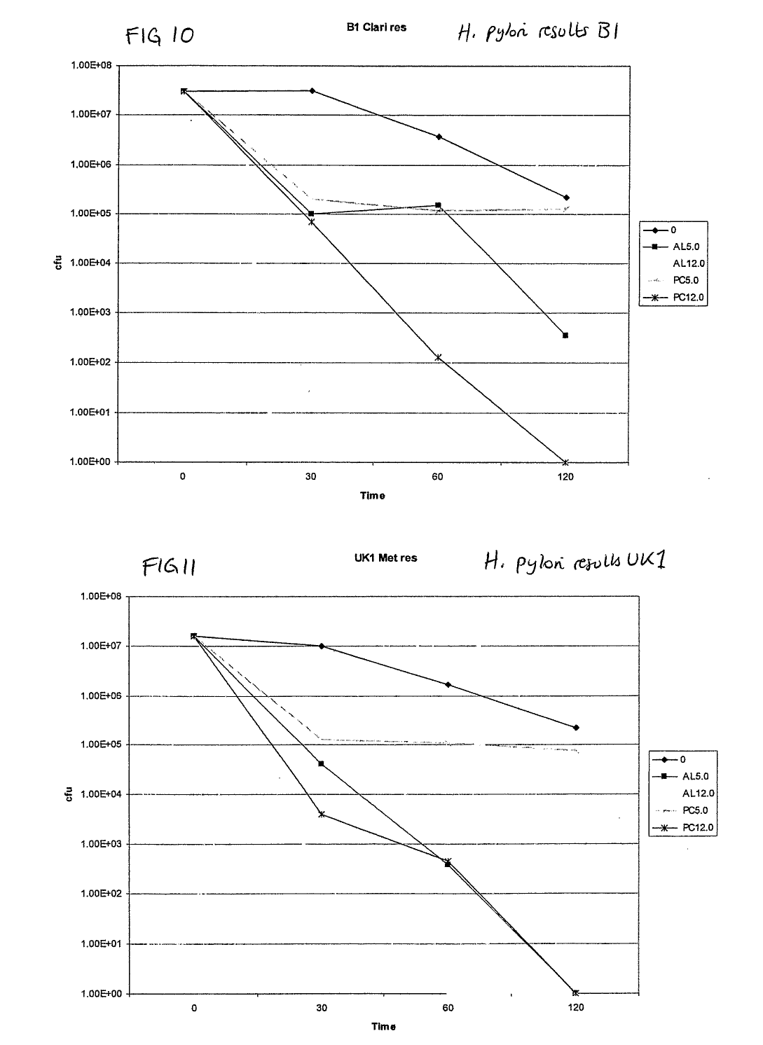

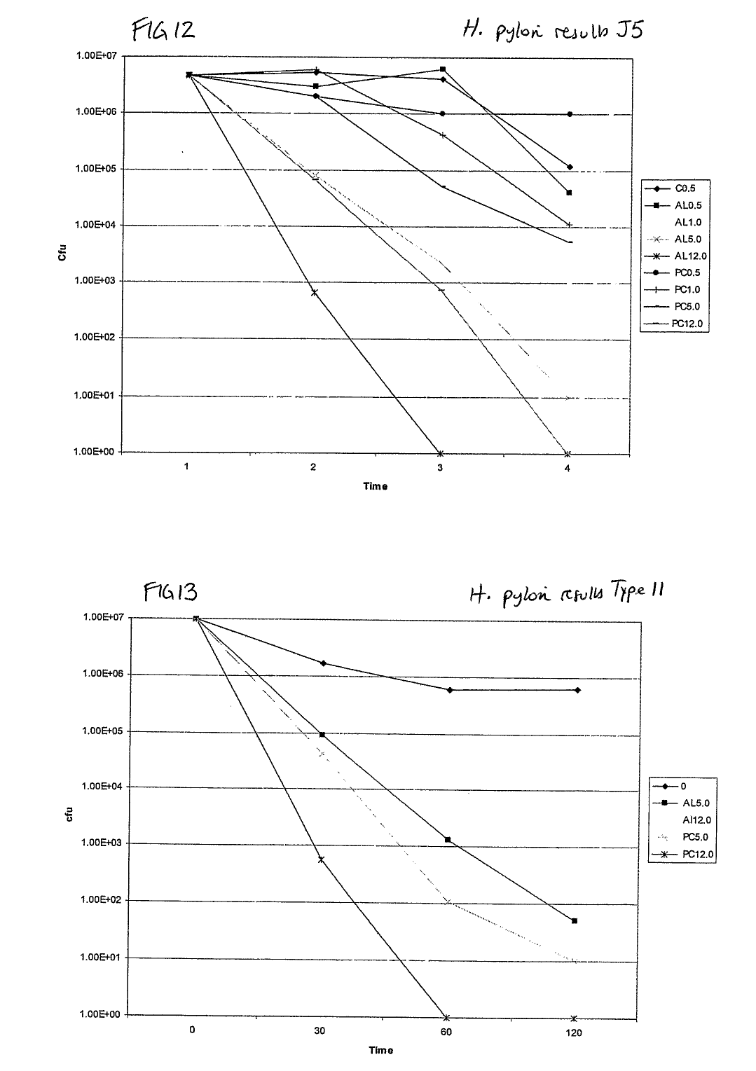

FIGS. 10 to 14 are graphs demonstrating the effects of the exemplified copper formulations relevant to H. pylori example 12, in which AL is used as an abbreviation for CuAL42, PC for CuPC33, and the concentrations being given in ppm, where 0 represents a control,

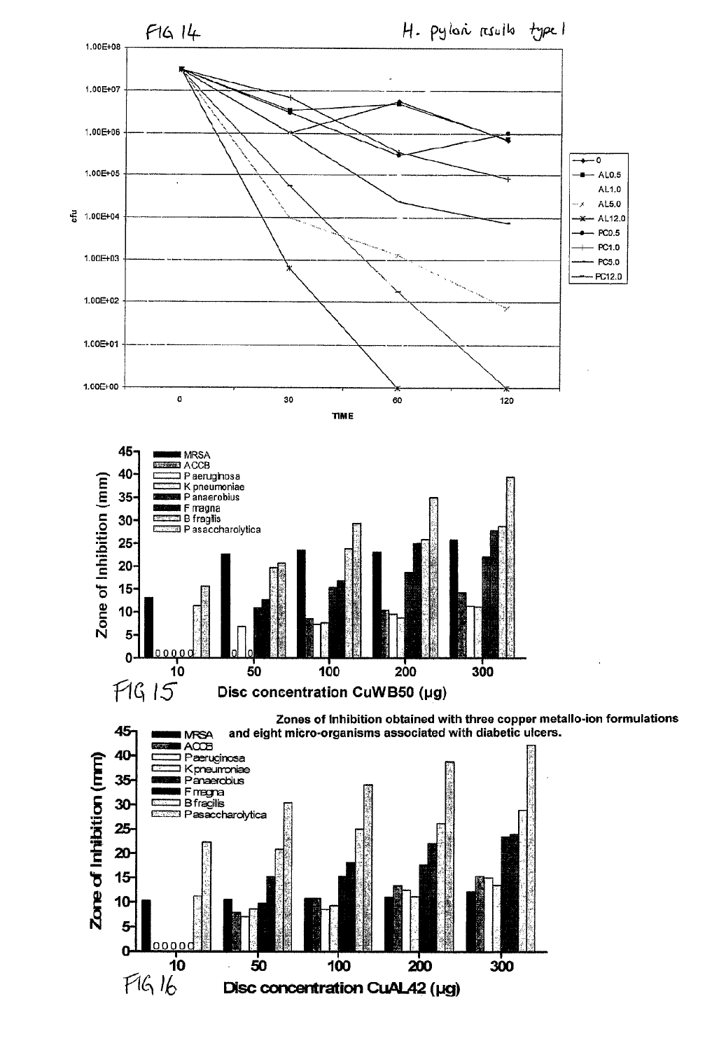

FIG. 15 shows the zones of inhibition obtained with the copper formulations exemplified coded CuAL42 and eight bacterial micro-organisms associated with diabetic foot ulcers,

FIG. 16 shows similar zones of inhibition as in FIG. 15, but using the copper antibacterial composition coded CuWB50,

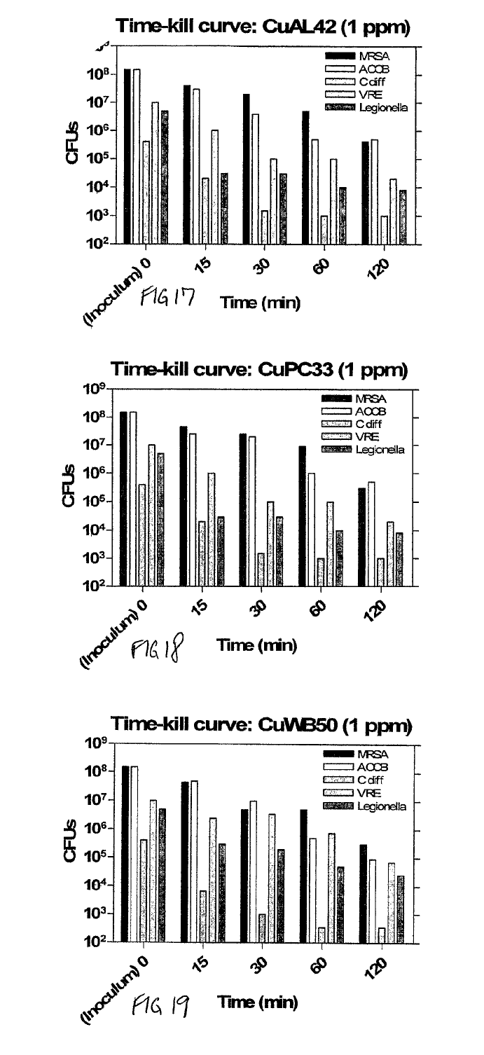

FIGS. 17 to 19 are plots representing time-kill curves of the three copper compositions at low dosage (1 ppm) against a variety of difficult to treat and/or antibiotic-resistant bacteria,

FIG. 10 shows the anti-MRSA activity of hand gel residues, relevant to example 13, where a gel type aqueous medium according to the invention (X-gel) is compared with a commercially available product,

FIG. 21 shows the disinfection of MRSA-contaminated UMF (ultra microfibre) cloths relevant to example 14, by impregnation with the three formulated copper antibacterial compositions, and

FIG. 22 is a comparison of hand gel cytotoxicity to the A431 human skin cell line, with other relevan products as explained in example 15.

### EXAMPLE 1

Introduction: Three copper metallo-ion formulations coded CuAL42, CuPC33 and CuWB50 obtained according to embodiments 1 to 8 of table 1 herein were tested for activity against the following target organisms: Methicillin resistant Staphylococcus aureus (MRSA); Acinetobacter calcoaceticus-baumanii (ACCB); Enterococcus sp. (vancomycin resistant; VRE); spores of Clostridium difficile; Legionella pneumophila.

The concentration of equivalent elemental copper in each of the three metallo-ion formulation stock solutions was 30.43 grams/litre, prior to dilution with distilled water. Each of the three copper formulations stock solutions were substantially diluted with deionised water and then tested at final post-dilution concentrations of 0.25, 0.5 and 1.0 part per million (ppm) of equivalent elemental copper against micro-organisms in logarithmic phase growth. The same compositions were also tested at 1 ppm against stationary phase micro-organisms.

Abbreviations: ACCB, Acinetobacter calcoaceticus-baumanii; MRSA, Methicillin resistant Staphylococcus aureus; PBS, phosphate-buffered saline; VRE, Enterococcus sp. (vancomycin resistant).

Materials and Methods: Blood agar, nutrient broth and BYCE medium were purchased from Oxoid Ltd (UK). MRSA, ACCB, and VRE were grown in pure culture on blood agar and a single colony transferred to nutrient broth and incubated with shaking for six hours at 37° C.

The six hour broth cultures (logarithmic phase cells) were then centrifuged to deposit the cells, the broth discarded and the bacterial cells washed and centrifuged three times using phosphate buffered saline at pH 7.2 (PBS). The final suspension was made in PBS and the viable cell count adjusted to the required inoculum for the experiments (1.5×10⁸). These cells were then exposed to the presently exemplified copper formulations at final concentrations of 0.25, 0.5 and 1.0 ppm.

Samples from these cultures were taken at 15, 30, 60 and 120 minutes and the viable count determined by the Miles and Misra technique. A control culture of PBS samples at 15 and 120 minutes was performed to ensure viability and stability of the inoculum.

The above examples were repeated at 1.0 ppm using stationary phase cells by taking cells from 24 hour agar plate cultures and suspending them directly into PBS after an initial PBS wash and an inoculum adjusted to 1.5×10⁸ cells/ml.

Clostridium difficile spore suspensions were made by suspending a five day culture of the organism on blood agar incubated anaerobically in 50:50 alcohol-saline. A Miles and Misra count was then performed on this suspension to determine the final concentration of viable spores and the inoculum finally adjusted to 5×10⁵ spores/ml for the tests.

Suspensions of Legionella pneumophila were made from five day cultures on BCYE medium in PBS and the viable count used to adjust the suspension to 5×10⁶ cells/ml.

All three copper formulations were tested against MRSA, ACCB and VRE using 6 hour cultures in nutrient broth as the challenge inocula.

Results: All three copper formulations—CuAL42 (Table A), CuPC33 (Table 2) and CuWB50 (Table 3) reduced bacterial numbers in a dose-dependent fashion. At a concentration of 1 ppm, all 3 copper formulations achieved around a three log inhibition of MRSA, ACCB and VRE. CuAL42 and CuPC33 gave a two log inhibition of C. difficile spores, whilst CuWB50 gave a three log inhibition of C. difficile spores.

CuAL42 and CuWB50 gave a two log inhibition of Legionella pneumophila and CuPC33 gave around three log inhibition.

As shown in Table 4, the inhibitory effect of the 3 copper formulations is similar for both log phase and stationary phase cells when using MRSA, ACCB and VRE.

In the other experiments the bacteria were grown in PBS and significant bacteriocidal effects were observed. As shown in Table 5, MRSA, ACCB and VRE were less sensitive to the bacteriocidal effects when grown in nutrient broth suggesting that the protein or other components are inhibiting the activity of the copper formulations. C. difficile and Legionella pneumophila were not tested in nutrient broth owing to technical difficulties in obtaining bacterial growth.

Discussion: The results presented here show that all 3 copper formulations are highly bacteriocidal to pathogenic bacteria at concentrations up to 1 ppm. However, this activity is somewhat neutralized when the bacteria are grown in nutrient broth suggesting that proteins or other components of the broth are reducing the efficacy of the copper formulations.

Interestingly, the copper formulations were highly active against growing bacteria and bacteria in stationary phase suggesting a cytotoxic effect on the bacterial cells rather than merely a static effect.

We have shown that MRSA grown in the presence of 0.1 ppm of CuAL42 for 10 days were 100% killed upon exposure to 1 ppm of CuAL42.

TABLE A
Time-kill curves with CuAL42
(copper sulphate/ammonium sulphate/sulphuric acid)

| | | | | |
|---|---|---|---|---|
| Concn (ppm) | 15 min | 30 min | 60 min | 120 min |

(a) MRSA (wild strain)

| | | | | |
|---|---|---|---|---|
| Control | 1.5 × 10⁸ | — | — | 1.5 × 10⁸ |
| 0.25 | 9 × 10⁷ | 9 × 10⁷ | 1 × 10⁷ | 2 × 10⁶ |
| 0.5 | 8 × 10⁷ | 2 × 10⁷ | 2 × 10⁷ | 1 × 10⁶ |
| 1.0 | 4 × 10⁷ | 2 × 10⁷ | 5 × 10⁶ | 4 × 10⁵ |

(b) Acinetobacter (wild strain)

| | | | | |
|---|---|---|---|---|
| Control | 1.5 × 10⁸ | — | — | 1.5 × 10⁸ |
| 0.25 | 9 × 10⁷ | 1 × 10⁷ | 2 × 10⁶ | 1 × 10⁶ |
| 0.5 | 7 × 10⁷ | 9 × 10⁶ | 1 × 10⁶ | 8 × 10⁵ |
| 1.0 | 3 × 10⁷ | 4 × 10⁶ | 5 × 10⁵ | 5 × 10⁵ |

(c) Clostridium difficile spore suspension

| | | | | |
|---|---|---|---|---|
| Control | 4 × 10⁵ | — | — | 4 × 10⁵ |
| 0.25 | 4 × 10⁵ | 4 × 10⁴ | 2.5 × 10⁴ | 2.5 × 10⁴ |
| 0.5 | 4 × 10⁵ | 3 × 10⁴ | 2 × 10⁴ | 2 × 10⁴ |
| 1.0 | 2.5 × 10⁴ | 2.5 × 10⁴ | 1.5 × 10⁴ | 16 × 10³ |

(d) Enterococcus (Vancomycin resistant, wild strain)

| | | | | |
|---|---|---|---|---|
| Control | 1 × 10⁷ | — | — | 10⁷ |
| 0.25 | 3 × 10⁵ | 3 × 10⁵ | 2 × 10⁵ | 2 × 10⁵ |
| 0.5 | 1 × 10⁵ | 5 × 10⁴ | 5 × 10⁴ | 2 × 10⁴ |
| 1.0 | 1 × 10⁵ | 1.5 × 10⁴ | 2.5 × 10³ | 1 × 10³ |

(e) Legionella pneumophila NCTC

| | | | | |
|---|---|---|---|---|
| Control | 5 × 10⁶ | — | — | 5 × 10⁶ |
| 0.25 | 2 × 10⁵ | 1 × 10⁵ | 6 × 10⁴ | 2.5 × 10⁴ |
| 0.5 | 1 × 10⁵ | 9.5 × 10⁴ | 7.5 × 10⁴ | 2.5 × 10⁴ |
| 1.0 | 1 × 10⁵ | 7.5 × 10⁴ | 7.5 × 10⁴ | 4 × 10⁴ |

TABLE 2
Time-kill curves with CuWB50
(copper sulphate/ammonium chloride/hyrdrochloric acid)

| | | | | |
|---|---|---|---|---|
| Concn (ppm) | 15 min | 30 min | 60 min | 120 min |

(a) MRSA (wild strain)

| | | | | |
|---|---|---|---|---|
| Control | 1.5 × 10⁸ | — | — | 1.5 × 10⁸ |
| 0.25 | 5 × 10⁷ | 9 × 10⁶ | 2 × 10⁶ | 1 × 10⁶ |
| 0.5 | 3 × 10⁷ | 8 × 10⁶ | 2 × 10⁶ | 8 × 10⁵ |
| 1.0 | 4.5 × 10⁷ | 5 × 10⁶ | 5 × 10⁶ | 3 × 10⁵ |

(b) Acinetobacter (wild strain)

| | | | | |
|---|---|---|---|---|
| Control | 1.5 × 10⁸ | — | — | 1.5 × 10⁸ |
| 0.25 | 9 × 10⁷ | 7 × 10⁷ | 1 × 10⁶ | 9 × 10⁵ |
| 0.5 | 9 × 10⁷ | 2 × 10⁷ | 9 × 10⁵ | 2 × 10⁵ |
| 1.0 | 5 × 10⁷ | 1 × 10⁷ | 5 × 10⁵ | 9 × 10⁴ |

(c) Clostridium difficile spore suspension

| | | | | |
|---|---|---|---|---|
| Control | 4 × 10⁵ | — | — | 4 × 10⁵ |
| 0.25 | 1.5 × 10⁴ | 6 × 10³ | 6 × 10² | 4 × 10² |
| 0.5 | 12 × 10³ | 3 × 10³ | 4.5 × 10² | 3.5 × 10² |
| 1.0 | 6.5 × 10³ | 1 × 10³ | 3.5 × 10² | 3.5 × 10² |

(d) Enterococcus (Vancomycin resistant, wild strain)

| | | | | |
|---|---|---|---|---|
| Control | 10⁷ | — | — | 10⁷ |
| 0.25 | 11 × 10⁶ | 8.5 × 10⁶ | 12 × 10⁵ | 12 × 10⁵ |
| 0.5 | 8.5 × 10⁶ | 6 × 10⁶ | 12 × 10⁵ | 7.5 × 10⁴ |
| 1.0 | 2.5 × 10⁶ | 3.5 × 10⁶ | 7.5 × 10⁵ | 7 × 10⁴ |

(e) Legionella pneumophila NCTC

| | | | | |
|---|---|---|---|---|
| Control | 5 × 10⁶ | — | — | 5 × 10⁶ |
| 0.25 | 8 × 10⁵ | 4 × 10⁵ | 2 × 10⁵ | 2 × 10⁵ |
| 0.5 | 3 × 10⁵ | 3 × 10⁵ | 1 × 10⁵ | 7.5 × 10⁴ |
| 1.0 | 3 × 10⁵ | 2 × 10⁵ | 5 × 10⁴ | 2.5 × 10⁴ |

TABLE 3
Time-kill curves with CuPC33
(copper sulphate/ammonium phosphate/phosphoric acid)

| | | | | |
|---|---|---|---|---|
| Concn (ppm) | 15 min | 30 min | 60 min | 120 min |

(a) MRSA (wild strain)

| | | | | |
|---|---|---|---|---|
| Control | 1.5 × 10⁸ | — | — | 1.5 × 10⁸ |
| 0.25 | 7 × 10⁷ | 2 × 10⁷ | 8 × 10⁶ | 1 × 10⁶ |
| 0.5 | 6 × 10⁷ | 2 × 10⁷ | 6 × 10⁶ | 8 × 10⁵ |
| 1.0 | 4.5 × 10⁷ | 2.5 × 10⁷ | 9 × 10⁶ | 3 × 10⁵ |

(b) Acinetobacter (wild strain)

| | | | | |
|---|---|---|---|---|
| Control | 1.5 × 10⁸ | — | — | 1.5 × 10⁸ |
| 0.25 | 9 × 10⁷ | 3 × 10⁷ | 1 × 10⁷ | 2 × 10⁶ |
| 0.5 | 5 × 10⁷ | 1 × 10⁷ | 8 × 10⁶ | 8 × 10⁵ |
| 1.0 | 2.5 × 10⁷ | 2 × 10⁷ | 1 × 10⁶ | 5 × 10⁵ |

(c) Clostridium difficile spore suspension

| | | | | |
|---|---|---|---|---|
| Control | 4 × 10⁵ | — | — | 4 × 10⁵ |
| 0.25 | 3.5 × 10⁴ | 1.5 × 10⁴ | 1.5 × 10⁴ | 1 × 10³ |
| 0.5 | 3.5 × 10⁴ | 2 × 10⁴ | 9.5 × 10³ | 2 × 10³ |
| 1.0 | 2 × 10⁴ | 1.5 × 10³ | 1 × 10³ | 1 × 10³ |

(d) Enterococcus (vancomycin resistant, wild strain)

| | | | | |
|---|---|---|---|---|
| Control | 1 × 10⁷ | — | — | 1 × 10⁷ |
| 0.25 | 1.4 × 10⁶ | 1.2 × 10⁶ | 1 × 10⁵ | 1 × 10⁵ |
| 0.5 | 1.4 × 10⁶ | 8.5 × 10⁵ | 1 × 10⁵ | 5 × 10⁴ |
| 1.0 | 1 × 10⁶ | 1 × 10⁵ | 1 × 10⁵ | 2 × 10⁴ |

(e) Legionella pneumophila NCTC

| | | | | |
|---|---|---|---|---|
| Control | 5 × 10⁶ | — | — | 5 × 10⁶ |
| 0.25 | 5 × 10⁴ | 5 × 10⁴ | 3 × 10⁴ | 2 × 10⁴ |
| 0.5 | 3 × 10⁴ | 3 × 10⁴ | 1 × 10⁴ | 1 × 10⁴ |
| 1.0 | 3 × 10⁴ | 3 × 10⁴ | 1 × 10⁴ | 8 × 10³ |

TABLE 4
Effect of 3 copper formulations (1 ppm) on stationary phase bacteria.

| | | | | |
|---|---|---|---|---|
| Inoculum | 15 min | 30 min | 60 min | 120 min |

CuAL42

| | | | | | |
|---|---|---|---|---|---|
| MRSA | 10⁸ | 6 × 10⁷ | 2 × 10⁷ | 4 × 10⁶ | 3 × 10⁵ |
| ACCB | 10⁸ | 5 × 10⁷ | 8 × 10⁶ | 7 × 10⁵ | 5 × 10⁵ |
| VRE | 10⁷ | 5 × 10⁶ | 3 × 10⁵ | 1 × 10⁵ | 7 × 10⁴ |

CuWB50

| | | | | | |
|---|---|---|---|---|---|
| MRSA | 10⁸ | 5 × 10⁷ | 2 × 10⁷ | 5 × 10⁶ | 2 × 10⁵ |
| ACCB | 10⁸ | 4 × 10⁷ | 1 × 10⁷ | 8 × 10⁵ | 1 × 10⁵ |
| VRE | 10⁷ | 2 × 10⁶ | 1 × 10⁶ | 7 × 10⁵ | 9 × 10⁴ |

CuPC33

| | | | | | |
|---|---|---|---|---|---|
| MRSA | 10⁸ | 4 × 10⁷ | 3 × 10⁷ | 1 × 10⁷ | 8 × 10⁵ |
| ACCB | 10⁸ | 3 × 10⁷ | 2 × 10⁷ | 6 × 10⁶ | 8 × 10⁵ |
| VRE | 10⁷ | 6 × 10⁶ | 5 × 10⁵ | 3 × 10⁵ | 8 × 10⁴ |

TABLE 5
Effect of 3 copper formulations
(1 ppm) on bacteria grown in nutrient broth.

| | | | |
|---|---|---|---|
| 15 min | 30 min | 60 min | 120 min |

CuAL42

| | | | | |
|---|---|---|---|---|
| MRSA | 8 × 10⁷ | 6 × 10⁷ | 6 × 10⁷ | 6 × 10⁷ |
| ACCB | 6 × 10⁷ | 3 × 10⁷ | 1 × 10⁷ | 8 × 10⁶ |
| VRE | 4 × 10⁷ | 3 × 10⁷ | 1 × 10⁷ | 1 × 10⁷ |

CuWB50

| | | | | |
|---|---|---|---|---|
| MRSA | 7 × 10⁷ | 2 × 10⁷ | 1 × 10⁷ | 4 × 10⁶ |
| ACCB | 4 × 10⁷ | 1 × 10⁷ | 1 × 10⁷ | 8 × 10⁶ |
| VRE | 5 × 10⁷ | 2 × 10⁷ | 8 × 10⁶ | 6 × 10⁶ |

CuPC33

| | | | | |
|---|---|---|---|---|
| MRSA | 5 × 10⁷ | 3 × 10⁷ | 1 × 10⁷ | 1 × 10⁷ |
| ACCB | 6 × 10⁷ | 4 × 10⁷ | 1 × 10⁷ | 9 × 10⁶ |
| VRE | 6 × 10⁷ | 2 × 10⁷ | 2 × 10⁷ | 7 × 10⁶ |

Initial inoculum = 10⁸ CFU/ml

### EXAMPLE 2

Introduction: The same three metallo-ion (copper) formulations coded CuAL42, CuPC33 and CuWB50 obtained according to embodiments 1 to 8 of table 1 herein, were investigated for their bactericidal properties when absorbed into an ultramicrofibre (UMF) cloth and then used to remove high level inocula of viable bacteria (MRSA, ACCB or C diff) from a common environmental hospital surface (laminated worktop surface).

Abbreviations: ACCB, Acinetobacter calcoaceticus-baumanii; C diff, Clostridium difficile (spores); MRSA, methicillin-resistant Staphylococcus aureus; PBS, phosphate buffered saline; ppm, parts per million; UMF, ultramicrofibre cloth.

Materials and Methods: The MRSA, ACCB and C diff (spores) organisms used in the study were clinical isolates.

The laminated surfaces were inoculated with 100 μl of phosphate buffered saline (PBS) containing 2×10⁶ colony forming units (cfu) of MRSA or ACCB or 3×10⁵ spores/ml of C diff spread with a sterile flat spreader over a 100 cm² area and allowed to dry. After drying the area was contact plated to ensure the viability of the inoculum.

The area was then cleaned with a UMF moistened to the recommended limit of wetness with sterile water (control) or with the respective copper formulation at a final concentration of 75 ppm.

The area was then contact plated again to assess the removal of the inoculum by the UMF. The UMF was then bagged in a mini-grip bag and left at room temperature for 16 hours to simulate travel to the laundry or static storage on the ward. After 16 hours the UMF was placed into 100 ml PBS and agitated in a Stomacher (Seward Ltd, UK) for 3 minutes at 250 rpm.

Viable counts were performed on the eluent and 10 ml of eluent centrifuged at 3500 rpm for 10 minutes and the deposit cultured onto blood agar.

The background count of the boards and the counts of PBS were tested for any environmental contamination. The results shown are the average of three separate runs.

Results: As shown in Table 6, contact plating revealed a heavy viable inoculum that was very effectively removed by the UMF. However, in the absence of copper formulations the bacteria remain viable on the UMF cloths. All three copper formulations killed 100% of Acinetobacter and C. difficile spores and a produced a four Log kill of MRSA. There were no recoverable Acinetobacter or the C. difficile bacteria from the Stomacher eluents of UMF-Cu formulation impregnated cloths.

Discussion: These studies investigated the ability of ultramicrofibre cloths to clean contaminated surfaces with and without copper-based anti-bacterial formulations. Whilst the UMF cloths were shown to be highly effective at removing bacteria from surfaces, the bacteria remain viable on the cloths for at least 16 hours. When the UMF cloths are pretreated with any of the 3 copper-based formulations, the cleaning efficacy was unchanged, but bacterial survival on the cloths was completely prevented for ACCB and C diff spores and was reduced by 4 Logs with MRSA. These results show that UMF cloths are highly efficacious for cleaning contaminated surfaces, but pretreatment of the cloths with copper-based anti-bacterial formulations according to examples of the present invention greatly reduces survival of these pathogenic bacteria on the cloths, which could be of immense benefit in hospitals and homes.

TABLE 6
Cleaning of contaminated surfaces with UMF cloths with and without
copper-based anti-bacterial formulations.

| | Cfu's detected | | | | | |
|---|---|---|---|---|---|---|
| Copper | with contact | | Stomacher eluent | | | |
| composition | plates | | from UMF/Cu | Board | | Inoculum |
| (75 ppm) and | Pre- | Post- | after 16 hr at room | surface | PBS | used per |
| bacteria used | clean | clean | temperature | control* | control** | 100 cm² |
| CuAL42 | | | | | | |
| MRSA | >500 | 0 | 6.6 × 10² | 0 | 0 | 2 × 10⁶ |
| ACCB | >500 | 0 | 0 | 0 | 0 | 2 × 10⁶ |
| CD spores | >500 | 0 | 0 | 0 | 0 | 3 × 10⁵ |
| CuPC33 | | | | | | |
| MRSA | >500 | 0 | 6.6 × 10² | 0 | 0 | 2 × 10⁶ |
| ACCB | >500 | 0 | 0 | 0 | 0 | 2 × 10⁶ |
| CD spores | >500 | 0 | 0 | 0 | 0 | 3 × 10⁵ |
| CuWB50 | | | | | | |
| MRSA | >500 | 0 | 3.3 × 10² | 0 | 0 | 2 × 10⁶ |
| ACCB | >500 | 0 | 0 | 0 | 0 | 2 × 10⁶ |
| CD spores | >500 | 0 | 0 | 0 | 0 | 3 × 10⁵ |
| Control UMF | | | | | | |
| MRSA | >500 | 0 | 2 × 10⁶ | — | — | 2 × 10⁶ |
| ACCB | >500 | 0 | 2 × 10⁶ | — | — | 2 × 10⁶ |
| CD spores | >500 | 0 | 3 × 10⁵ | — | — | 3 × 10⁵ |

*checks for environmental contaminants.
**sterility check of PBS.

### EXAMPLE 3

Introduction: The presence in hospitals of antibiotic resistant bacteria such as methicillin-resistant Staphylococcus aureus (MRSA) and vancomycin-resistant Enterococci, and spores of Clostridium difficile that are very difficult to destroy is an increasingly serious problem. These organisms can also colonize nurse's uniforms and this represents a method by which the bacteria can be spread around hospitals and into the general environment.

Therefore, the present example was undertaken to determine whether the copper-based metallo-ion formulation called CuWB50 (as defined herein) already shown herein to be active against MRSA, Acinetobacter sp., E. coli and Clostridium difficile in vitro has activity in a model washing system with and without Ariel™ biological detergent.

Abbreviations: C diff, Clostridium difficile; MRSA, methicillin-resistant Staphylococcus aureus; ppm, parts per million;

Material and Methods: The MRSA and C diff (spores) organisms used in the study were clinical isolates. The Stomacher® 400 Circulator was purchased from Seward Ltd (UK).
1. Washing protocol using Ariel detergent with or without the embodiment of copper formulation referred to herein as CuWB50. Swatches of nursing uniform material (100 cm²) were contaminated with MRSA or C diff spores and allowed to dry at room temperature for 3 hours. Each swatch was added to a plastic bag containing 20 ml of water with Ariel detergent added at the Manufacturer's recommended concentration with or without 200 ppm of CuWB50. Each swatch was processed in a circulating Stomacher for 15 min at 240 rpm at room temperature to simulate a low temperature wash cycle. After washing, 2 ml of the eluent was mixed with 2 ml of calcium-rich Ringer's solution to neutralize any CuWB50 carry over. Neutralized eluent (0.1 ml) was then spread onto blood agar plates and incubated overnight at 37° C. in air (MRSA) or anaerobically (C diff spores) when the colonies were counted on duplicate plates.
2. Washing protocol with CuWB50 added to the rinse cycle. Swatches of nursing uniform material (100 cm²) were contaminated with MRSA or C diff spores and allowed to dry at room temperature for 3 hours. Each swatch was added to a plastic bag containing 20 ml of water and was then processed in a circulating Stomacher for 15 min at 240 rpm at room temperature to simulate a low temperature wash cycle. After the water only wash cycle, the water was replaced with 20 ml of water containing 200 ppm of CuWB50 and then processed again in the Stomacher for 5 min to simulate a rinse cycle. 2 ml of the eluent was mixed with 2 ml of calcium-rich Ringer's solution to neutralize any CuWB50 carry over. Neutralized eluent (0.1 ml) was then spread onto blood agar plates and incubated overnight at 37° C. in air (MRSA) or anaerobically (C diff spores) when the colonies were counted on duplicate plates.

Results: As shown in Table 7, post-wash recovery of MRSA and C. diff was reduced by 2-3 logs from the original inoculum levels when the nursing uniform material swatches were washed with Ariel detergent alone. In contrast, there was a complete 6 log kill when the wash contained Ariel with 200 ppm of CuWB50.

When the nursing uniform material swatches were washed in water alone, the post-wash recovery of MRSA and C diff was only slightly reduced by less than 1 log in each case as shown in Table 8. However, after a 5 minute rinse in water containing 200 ppm of CuWB50 all of the remaining organisms were killed and no colonies were observed.

Discussion: We have used a model washing system with swatches of nursing uniform material contaminated with methicillin-resistant Staphylococcus aureus (MRSA) or Clostridium difficile spores (C diff) to assess the anti-microbial effects of washing with Ariel biological detergent with and without CuWB50 or adding CuWB50 to a rinse cycle.

The results show that while Ariel reduces bacterial contamination by 2-3 logs, CuWB50 is 100% effective in removing/killing bacteria when added to either the washing or rinse cycles.

Addition of copper-based metallo-ion formulations in accordance with the present invention to hospital and home laundry may be an economic and effective way to sterilize clothing.

TABLE 7
Washing protocol using Ariel detergent with or without CuWB50.

| | Initial | Recovery post Ariel | Recovery post Ariel + |
|---|---|---|---|
| Organism | inoculum | wash | CuWB50 wash |
| MRSA | 2 × 10⁶ | 6.0 × 10³ | 0 |
| C diff spores | 1 × 10⁶ | 4.2 × 10⁴ | 0 |

TABLE 8
Washing protocol with CuWB50 added to the rinse cycle.

| | Initial | Recovery post initial | Recovery post rinse with |
|---|---|---|---|
| Organism | inoculum | water wash | CuWB50 (200 ppm) |
| MRSA | 2 × 10⁵ | 8.0 × 10⁴ | 0 |
| C diff spores | 1 × 10⁵ | 6.0 × 10⁴ | 0 |

### EXAMPLE 4

Introduction: Diabetic ulcers represent a serious medical condition that is difficult to treat, particularly when infected with anaerobic or antibiotic resistant bacteria. Diabetic foot ulcers are frequently disabling and can lead to amputation of toes, feet and even legs.

Infection of diabetic ulcers commonly occurs with one or more of the following organisms:

Methicillin-resistant Staphylococcus aureus (MRSA), Pseudomonas aeruginosa, A calcoaceticus-baumanii, Klebsiella pneumoniae, Bacteroides fragilis, Porphyromonas asaccharolytica, Finegoldia magna, Peptostreptococcus anaerobius [1-3].

The aim of the present example was to determine whether three copper-based metallo-ion formulations as defined herein called CuAL42, CuPC33 and CuWB50 that have been shown to be active against MRSA, Acinetobacter sp., E. coli and Clostridium difficile would also have activity against the diabetic ulcer-related organisms listed above.

Materials and Methods: The organisms used in the study were clinical isolates. The names of the strains and the abbreviated name used in Table 1 are as follows: methicillin-resistant Staphylococcus aureus (MRSA), A calcoaceticus-baumanii (ACCB), Pseudomonas aeruginosa (P aerug), Klebsiella pneumoniae, (K pneum), Bacteroides fragilis (B fragilis), Porphyromonas asaccharolytica (P asacch), Finegoldia magna (F magna), Peptostreptococcus anaerobius (P anaerob).

A MacFarland 0.5 ml standard suspension was made of each of these organisms in buffered isotonic saline. A swab was dipped into the bacterial suspension and then plated onto blood agar using a rotary plater in order to develop a lawn of bacteria on the agar plates.

Paper discs containing various concentrations of CuAL42, CuPC33 and CuWB50 (calculated as μg of elemental copper per disc) were placed onto the agar surface and the plates incubated anaerobically in a Don Whitley Anaerobic Workstation at 37° C. for 24 hours (anaerobic bacteria) or at 37° C. in air for 24 hours (aerobic bacteria).

Zones of inhibition were measured using electronic callipers and recorded. The results shown in Table 9 are of tests made in duplicate.

Results: As shown in Table 9 and FIGS. 15 and 16, all 3 copper formulations were consistently highly active against all 8 micro-organisms tested at concentrations above 100 μg of elemental copper.

Some slight variability was seen in sensitivity of certain bacteria to the 3 different formulations e.g. A calcoaceticus-baumanii was more sensitive to CuAL42 and CuPC33 than CuWB50 at 50 μg and K pneumoniae was only sensitive to CuAL42 at 50 μg.

MRSA, B fragilis and P asaccharolytica were sensitive to all 3 copper formulations at 10 μg, the lowest concentration tested.

Discussion: It is clear that concentrations of all 3 copper formulations above 100 μg of elemental copper on the discs produced significant zones of inhibition for all 8 organisms. These results are consistent with studies using tube dilution tests where at least 75 μg of the copper formulations were required to inhibit bacteria in the presence of nutrient broth, and also studies with microfibre cloths where 75 μg of copper completely killed bacteria on the stored cloths. Both aerobic and anaerobic bacteria commonly found in infected ulcers in diabetic patients are susceptible to levels of 100 μg or more of copper as revealed by wide zones of inhibition in these disc tests. There were some differences in activity with different copper formulations and certain organisms but these were modest.

The results suggest that washes, soaps and gels containing one or more of the exemplified copper formulations may be useful in the treatment of diabetic ulcers by virtue of their ability to kill bacteria that are responsible for the maintenance and spread of diabetic ulcers, and an ability to accelerate the skin healing process.

TABLE 9
Zones of Inhibition obtained with three copper metallo-ion formulations
and eight micro-organisms associated with diabetic ulcers.

| | Disc concentration | MRSA¹ | ACCB | P aerug | K pneum | B fragilis | P asacch | F magna | P anaerob |
|---|---|---|---|---|---|---|---|---|---|
| Compound | (μg) | Diameter of Zones of Inhibition (mm) | | | | | | | |
| CuAL42 | 10 | 10.39 | 0 | 0 | 0 | 11.3 | 22.43 | 0 | 0 |
| | 50 | 10.62 | 7.95 | 7.07 | 8.69 | 20.98 | 30.55 | 15.34 | 9.81 |
| | 100 | 10.83 | 10.80 | 8.62 | 9.38 | 25.17 | 34.26 | 18.30 | 15.35 |
| | 200 | 11.12 | 13.47 | 12.50 | 11.24 | 26.37 | 39.11 | 22.34 | 17.65 |
| | 300 | 12.33 | 15.51 | 15.06 | 13.55 | 29.18 | 42.46 | 24.16 | 23.74 |
| CuPC33 | 10 | 9.80 | 0 | 0 | 0 | 12.91 | 18.30 | 7.2 | 0 |
| | 50 | 10.00 | 7.77 | 7.58 | 0 | 18.10 | 22.29 | 14.89 | 11.98 |
| | 100 | 12.28 | 10.05 | 9.49 | 7.89 | 24.89 | 30.66 | 19.21 | 15.80 |
| | 200 | 13.43 | 12.05 | 12.42 | 9.98 | 25.91 | 40.96 | 23.10 | 20.00 |
| | 300 | 23.61 | 14.52 | 13.99 | 13.70 | 27.28 | 42.64 | 25.11 | 26.44 |
| CuWB50 | 10 | 13.14 | 0 | 0 | 0 | 11.35 | 15.57 | 0 | 0 |
| | 50 | 22.69 | 0 | 6.83 | 0 | 19.66 | 20.74 | 12.72 | 10.91 |
| | 100 | 23.59 | 8.56 | 7.37 | 7.71 | 23.93 | 29.54 | 16.76 | 15.36 |
| | 200 | 23.19 | 10.37 | 9.53 | 8.84 | 25.88 | 35.12 | 25.11 | 18.65 |
| | 300 | 25.82 | 14.21 | 11.29 | 11.05 | 28.80 | 39.52 | 28.03 | 22.13 |

¹Abbreviations are shown in Materials and Methods.

### EXAMPLE 5

Introduction: There is little current evidence that the recommended time/temperature relationships for laundry as given in HSG(95)18 are efficacious for organisms that are of a particular concern in nosocomial infection. Furthermore, there is little scientific support for these laundry conditions. Consequently, the present example was undertaken to define the conditions that lead to reduction of contaminated linens under cold wash conditions.

A cold wash cycle was considered the most demanding test of the anti-microbial copper formulations. In addition, it seems likely that increasingly high energy costs will lead to the use of lower wash temperatures both in industrial and home washing—particularly if a sufficiently easy-to-use and economical anti-microbial product that is also “kind” to fabrics can be developed.

This study describes a study of decontamination of laundry using fabric that has been contaminated with marker micro-organisms. The test materials are a metallo-ion (copper) formulation called CuWB50 and two commercially available washing detergents (designated A and P) in an Electrolux washing machine using a low temperature (18° C.) wash.

Abbreviations: ACCB, Acinetobacter sp.; BSA, bovine serum albumin; cfu, Colony forming units; MRSA, methicillin-resistant Staphylococcus aureus; PBS, phosphate-buffered saline;

Materials and Methods: Commercially available swatches of typical hospital quality uniform fabric were supplied by Carrington Career & Work wear Ltd (UK). The composition of the swatches is a 67% polyester/33% cotton blend with a fabric weight of 195 g/m².

A commercial washing machine, upgraded with the new Claris control system, was purchased from Electrolux. The Claris control system provides the researcher with complete flexibility to control time and temperature of each wash cycle. The Claris system also provides electronic data output recording the specifications of each wash cycle. The Stomacher® 400 Circulator was purchased from Seward Ltd (UK).

The washing detergents A and P were purchased from a local supermarket. Bovine serum albumin (BSA) was purchased from Sigma-Aldrich. All microbiological reagents and agar plates were purchased from Oxoid Ltd (UK). PBS and BSA were purchased from Sigma.

The swatches were each contaminated with an inoculum of 2×10⁸ bacteria of clinical isolates of methicillin-resistant Staphylococcus A (MRSA) or multi-resistant Acinetobacter sp. (ACCB) in a volume of 2 ml of PBS containing 7% BSA. The swatches were dried at room temperature prior to use in the washing studies.

The swatches were attached to ballast linen to give a final weight of 5 kg per cold water wash in order to mimic a normal wash load in 15 litres of water with a standard wash time of 15 minutes. Six washing conditions were assessed with both bacterial strains: 1. Water alone; 2. Water+Detergent A; 3. Water+Detergent P; 4, Water+CuWB50; 5. Water+Detergent A+CuWB50; 6. Water+Detergent P+CuWB50. The concentration of CuWB50 was 100 ppm and a single gelule of detergent A (50 ml) or detergent P (25 g) was used unless otherwise stated. At the end of each wash 1 litre of post-wash machine water was collected and 100 ml was centrifuged and the bacterial pellet tested for colony-forming units (cfu).

Washed contaminated swatches (n=3), control uncontaminated (clean) swatches (n=2; used to assess transfer of bacteria from contaminated swatches during washing), and contaminated, unwashed swatches to give an actual measure of bacterial contamination as cfu (as opposed to the original inoculum, thus controlling for loss of viability of the organism during the drying period), were placed individually in plastic bags with 20 ml of PBS and massaged in a Stomacher for 15 minutes at room temperature.

Decimal dilutions of the resulting Stomacher bacterial suspensions and also post-wash machine water were plated onto duplicate agar plates and the number of cfu was counted following a 24 hr incubation period at 37° C.

Results: In each of the following Tables the results are presented for (i) control contaminated swatches=initial bacterial inoculum in cfu, (ii) post-wash contaminated swatches=remaining bacterial cfu on the contaminated swatches after washing, (iii) post-wash machine eluent=cfu of free bacteria in the wash water at the end of the 15 min wash cycle, and (iv) post-wash clean swatches=bacterial cfu on uncontaminated swatches after washing (indicates bacterial transfer during washing).

The results in Table 10 show that cold water washing produced a modest decrease in the number of cfu on the contaminated swatches—a 2 Log reduction with ACCB and a 4 Log reduction with MRSA. The cfu of ACCB and MRSA in the machine eluent post-wash was similar for both bacteria and the transfer of bacterial cfu to the clean swatches was around 2 Logs lower than the level of cfu remaining on the contaminated swatches after washing. These results indicate that the 15 minute wash cycle with cold water alone can dislodge some bacteria from the contaminated swatches into the water and that some of these free bacteria can attach onto the clean swatches during the washing cycle.

The results in Table 11 show that a cold water wash with either detergent produces a modest decrease in the number of Acinetobacter cfu on the contaminated swatches—slightly greater than a 2 Log reduction with detergent A and slightly less than a 2 Log reduction with detergent P. The cfu of ACCB in the machine eluent post-wash was slightly greater for detergent A than P, although the initial inoculum was also slightly higher in the example with detergent A. The transfer of bacterial cfu to the clean swatches was around 2 Logs lower than the level of cfu remaining on the contaminated swatches after washing. These results indicate that the 15 minute wash cycle with cold water and detergents can dislodge some Acinetobacter from the contaminated swatches into the water and that some of the free bacteria can attach onto the clean swatches during the washing cycle. However, the results were not very different from those shown in Table 10 with water alone indicating that these detergents have little anti-bacterial activity against Acinetobacter.

The results in Table 12 show that a cold water wash with both detergents produces a substantial 5 to 6 Log decrease in the number of MRSA cfu on the contaminated swatches, suggesting that both detergents have a strong anti-bacterial effect against MRSA. The levels of cfu of MRSA in the machine eluent post-wash and transferred to the clean swatches were very low supporting the view that the detergents have a strong anti-bacterial effect with MRSA. These results indicate that both detergents have a strong anti-bacterial effect on MRSA that was not seen with Acinetobacter (Table 11).

The results in Table 13 show that a cold water wash with CuWB50 alone is highly effective at reducing bacterial contamination over a wide concentration range. Acinetobacter is more sensitive to CuWB50 and is completely killed at concentrations of 100 and 15 ppm. At CuWB50 concentrations of 1 to 10 ppm there is still a considerable anti-bacterial effect with a 3 to 5 Log reduction of Acinetobacter cfu. At almost all CuWB50 concentrations, Acinetobacter was unable to survive in the machine eluent or to be transferred to the clean swatches. CuWB50 was also effective against MRSA producing a 4 to 5 Log kill at concentrations from 1 to 100 ppm. As with Acinetobacter, very few MRSA cfu were detected in the machine eluent or on clean swatches at any concentration of CuWB50. These results show that both bacterial strains are highly sensitive to CuWB50 with Acinetobacter being somewhat more sensitive than MRSA.

The results in Table 14 clearly show that 100 ppm of CuWB50 combined with either detergent A or P leads to complete killing of both Acinetobacter and MRSA with no detectable cfu in any of the post wash samples. The results in Table 11 show that either detergent alone has little bacteriocidal effect on Acinetobacter (2 Log kill), whilst the results in Table 13 show that 100 ppm of CuWB50 completely kills Acinetobacter which explains the results above.

The results in Table 12 show that both detergents alone were quite effective against MRSA producing a 5 Log kill, and the results in Table 13 show that CuWB50 is also quite effective against MRSA (5 Log kill). Therefore, the results above suggest an additive effect of the detergents with CuWB50 leading to complete kill of MRSA.

The results shown in Table 15 confirm those in Table 14 showing that CuWB50 at 100 pm combined with detergent A completely kills both Acinetobacter and MRSA under cold wash conditions. Furthermore, the results in Table 15 show that detergent A and CuWB50 at concentrations down to as low as 5 ppm are highly effective at killing both bacteria. MRSA is also completely killed by detergent A with CuWB50 at 2 ppm, whilst Acinetobacter was less sensitive to this concentration with only a 2 Log kill. These results show that CuWB50 at concentrations of 5 ppm and higher combined with detergent A forms a potent anti-bacterial combination even at using a low wash temperature.

Discussion: The effect of a biocidal copper compound, CuWB50, on cold water washing of MRSA- or Acinetobacter-contaminated swatches of nurse's uniform fabric with and without 2 commercial washing detergents was assessed using an industrial Electrolux washing machine. Washing with cold water alone produced a 2-3 Log reduction in MRSA and ACCB cfu on the contaminated swatches (Table 10), but the released bacteria were detected in the machine post-wash effluent and on the sterile swatches.

The two commercial detergents used alone were more effective at removing MRSA (5-6 Log reduction in cfu; Table 12) than ACCB (1-2 Log reduction in cfu; Table 11) from the contaminated swatches. In both cases, live bacteria were detected in the machine post-wash effluent and on the sterile swatches, but the numbers of bacteria recovered were reduced in the case of MRSA suggesting a modest anti-bacterial effect of the detergents on this bacterial strain. As shown in Table 13, CuWB50 alone killed ACCB completely at 100 and 15 ppm and reduced cfu by 3-4 Logs at concentrations as low as 1 ppm. CuWB50 alone reduced MRSA cfu by 4-5 Logs at 1-100 ppm. In both cases, the number of bacteria recovered in the machine post-wash effluent and on the sterile swatches was substantially reduced indicating an anti-bacterial effect of CuWB50 alone in the cold water wash even at low concentrations.

CuWB50 at 100 ppm combined with either detergent resulted in a 100% kill of both ACCB and MRSA on contaminated swatches, in the machine post-wash effluent and on the sterile swatches (Table 14). Since 100 ppm of CuWB50 was not completely effective alone against MRSA (Table 13) and both detergents showed some variability in their ability to kill MRSA (Table 12), there is clearly an additive effect leading to complete decontamination with the two products together. ACCB was relatively resistant to both detergents alone (Table 11), but was very sensitive to CuWB50 (Table 13) and the combination of CuWB50 with either detergent resulted in complete killing of ACCB.

In fact, the combination of CuWB50 and detergent A was very effective at all concentrations of CuWB50 (2-100 ppm) against MRSA and 5-100 ppm of CuWB50 against ACCB. In all cases, no live bacteria were recovered in the machine post-wash effluent or on the sterile swatches.

In conclusion, these results suggest that cold water washing of nurse's uniforms with detergents alone is unlikely to be effective in removing all bacterial contamination. The addition of as little as 5-10 ppm of CuWB50 with either detergent using a cold water wash resulted in complete disinfection of MRSA- and ACCB-contaminated swatches and the machine post-wash effluent and the sterile swatches. Since a 10 ppm concentration of CuWB50 was achieved by adding just 5 ml of the formulated composition stock solution to a 15 litre wash and considering the high levels of bacterial contamination on the swatches used in these experiments (around 10⁸ cfu), the results suggest that addition of CuWB50 to machine washes with normal amounts of commercial washing detergents could help significantly to reduce bacterial contamination in all hospital laundry. Although C. difficile spores were not tested, our results herein suggest that C. difficile spores would also be effectively decontaminated by a CuWB50/detergent combination.

TABLE 10
The effect of cold water washing alone on the removal of
Acinetobacter (ACCB) and MRSA from contaminated swatches.

| | Contaminated | Post-wash contaminated | Machine post- | Post-wash clean |
|---|---|---|---|---|
| | swatches cfu | swatches cfu | wash eluent cfu | swatches cfu |
| ACCB | | | | |
| Expt 1 | 1.2 × 10⁸ | 6.0 × 10⁵; 1.6 × 10⁵; 3.0 × 10⁴ | 4.9 × 10³ | 8.0 × 10²; 0 |
| Expt 2 | 7.2 × 10⁶ | 1.2 × 10⁵; 7.4 × 10⁴; 6.0 × 10⁴ | 1.5 × 10⁴ | 3.8 × 10³; 2.8 × 10³ |
| Expt 3 | 1.2 × 10⁷ | 1.0 × 10⁵; 4.2 × 10⁴; 4.5 × 10⁴ | 0 | NT; NT |
| Average | 4.6 × 10⁷ | 1.4 × 10⁵ | 6.6 × 10³ | 1.9 × 10³ |
| MRSA | | | | |
| Expt 1 | 9.0 × 10⁷ | 1.4 × 10⁴; 1.0 × 10⁵; 9.2 × 10⁵ | 2.4 × 10³ | 6.0 × 10²; 8.0 × 10² |
| Expt 2 | 2.6 × 10⁸ | 2.4 × 10³; 3.6 × 10³; 2.4 × 10³ | 8.2 × 10³ | 1.2 × 10³; 1.0 × 10³ |
| Expt 3 | 2.5 × 10⁸ | 1.9 × 10³; 2.4 × 10⁴; 1.6 × 10⁴ | 0 | NT; NT |
| Average | 2 × 10⁸ | 1.5 × 10⁵ | 3.5 × 10³ | 9 × 10² |

NT = Not tested

TABLE 11
The effect of cold water washing with detergents A or P on the removal of
Acinetobacter from contaminated swatches.

| | Contaminated | Post-wash contaminated | Machine post- | Post-wash clean |
|---|---|---|---|---|
| | swatches cfu | swatches cfu | wash eluent cfu | swatches cfu |
| Detergent A | | | | |
| Expt 1 | 1.1 × 10⁷ | 1.6 × 10⁵; 2.3 × 10⁵; 1.7 × 10⁵ | 5.9 × 10⁴ | 2.0 × 10³; 1.6 × 10³ |
| Expt 2 | 2.1 × 10⁸ | 3.8 × 10⁵; 2.4 × 10⁵; 4.8 × 10⁵ | 2.8 × 10⁴ | 3.6 × 10³; 2.8 × 10³ |
| Expt 3 | 5.8 × 10⁶ | 1.0 × 10⁵; 1.1 × 10⁵; 9.8 × 10⁴ | 5.8 × 10¹ | 0; 0 |
| Average | 7.6 × 10⁷ | 2.2 × 10⁵ | 2.9 × 10⁴ | 1.7 × 10³ |
| Detergent P | | | | |
| Expt 1 | 6.6 × 10⁶ | 2.1 × 10⁵; 1.6 × 10⁴; 2.1 × 10⁵ | 7.4 × 10² | 1.2 × 10³; 1.6 × 10³ |
| Expt 2 | 1.9 × 10⁷ | 4.0 × 10⁵; 3.4 × 10⁵; 3.6 × 10⁵ | 1.3 × 10³ | 3.0 × 10³; 1.6 × 10³ |
| Expt 3 | 2.6 × 10⁶ | 1.8 × 10⁴; 2.8 × 10⁴; 3.8 × 10⁴ | 5.4 × 10² | 6.0 × 10²; 5.0 × 10² |
| Average | 9.4 × 10⁶ | 1.8 × 10⁵ | 8.6 × 10² | 1.4 × 10³ |

TABLE 12
The effect of cold water washing with detergents A or P on the removal of MRSA
from contaminated swatches.

| | Contaminated | Post-wash contaminated | Machine post- | Post-wash clean |
|---|---|---|---|---|
| | swatches cfu | swatches cfu | wash eluent cfu | swatches cfu |
| Detergent A | | | | |
| Expt 1 | 1.9 × 10⁸ | 1.2 × 10³; 8.0 × 10²; 1.0 × 10³ | 1.2 × 10³ | 0; 2.0 × 10² |
| Expt 2 | 8.0 × 10⁷ | 2.0 × 10³; 3.2 × 10³; 2.6 × 10³ | 2.4 × 10³ | 0; 0 |
| Expt 3 | 1.0 × 10⁷ | 0; 0; 0 | 0 | 0; 0 |
| Expt 4 | 9.8 × 10⁶ | 0; 0; 0 | 0 | 0; 0 |
| Expt 5 | 7.4 × 10⁷ | 0; 0; 0 | 0 | 0; 0 |
| Average | 7.3 × 10⁷ | 7.2 × 10² | 7.2 × 10² | 2.0 × 10¹ |
| Detergent P | | | | |
| Expt 1 | 1.3 × 10⁸ | 0; 0; 0 | 1.5 × 10² | 0; 0 |
| Expt 2 | 2.9 × 10⁷ | 8.0 × 10²; 8.0 × 10²; 4.0 × 10² | 1.0 × 10¹ | 1.0 × 10³; 1.2 × 10³ |
| Expt 3 | 8.8 × 10⁷ | 0; 0; 0 | 0 | 0; 0 |
| Expt 4 | 2.0 × 10⁸ | 0; 0; 0 | 0 | 0; 0 |
| Expt 5 | 2.0 × 10⁸ | 0; 0; 0 | 0 | 0; 0 |
| Average | 1.3 × 10⁸ | 1.3 × 10² | 3.2 × 10¹ | 2.2 × 10² |

TABLE 13
The effect of cold water washing with the anti-bacterial copper formulation CuWB50 on
the removal of Acinetobacter (ACCB) and MRSA from contaminated swatches.

| CuWB50 | Contaminated | Post-wash contaminated | Machine post- | Post-wash clean |
|---|---|---|---|---|
| (ppm) | swatches cfu | swatches cfu | wash eluent cfu | swatches cfu |
| ACCB | | | | |
| 100 (n = 5)* | 4.8 × 10⁷ | 0 | 0 | 0 |
| 15 (n = 2) | 1.5 × 10⁷ | 0 | 0 | 0 |
| 10 (n = 1) | 4.1 × 10⁷ | 9.3 × 10² | 0 | 0 |
| 5 (n = 2) | 4.5 × 10⁷ | 1.4 × 10³ | 1.0 × 10¹ | 0 |
| 1 (n = 1) | 8.8 × 10⁶ | 1.5 × 10³ | 0 | 0 |
| MRSA | | | | |
| 100 (n = 5) | 1.9 × 10⁸ | 1.9 × 10³ | 6.2 × 10⁰ | 0 |
| 15 (n = 2) | 1.6 × 10⁸ | 1.8 × 10² | 0 | 0 |
| 10 (n = 1) | 2.5 × 10⁸ | 3.0 × 10³ | 2.0 × 10² | 0 |
| 5 (n = 2) | 8.7 × 10⁷ | 6.7 × 10³ | 2.1 × 10³ | 0 |
| 1 (n = 1) | 2.1 × 10⁷ | 9.3 × 10² | 0 | 0 |

*All results are the average of each set of experiments (number of experiments = n).

TABLE 14
The effect of cold water washing with CuWB50 (100 ppm) and 2 detergents (A and P) on
the removal of Acinetobacter (ACCB) and MRSA from contaminated swatches.

| CuWB50 100 ppm | Contaminated | Post-wash contaminated | Machine post- | Post-wash clean |
|---|---|---|---|---|
| plus . . . | swatches cfu | swatches cfu | wash eluent cfu | swatches cfu |
| ACCB | | | | |
| Detergent A* | 8.2 × 10⁶ | 0 | 0 | 0 |
| Detergent P | 1.2 × 10⁷ | 0 | 0 | 0 |
| MRSA | | | | |
| Detergent A | 3.9 × 10⁵ | 0 | 0 | 0 |
| Detergent P | 1.2 × 10⁷ | 0 | 0 | 0 |

*The results shown are average cfu for duplicate experiments.

TABLE 15
The effect of cold water washing with various concentrations of CuWB50 and detergent A
on the removal of Acinetobacter (ACCB) and MRSA from contaminated swatches.

| CuWB50 | Contaminated | Post-wash contaminated | Machine post- | Post-wash clean |
|---|---|---|---|---|
| (ppm) | swatches cfu | swatches cfu | wash eluent cfu | swatches cfu |
| ACCB | | | | |
| 50 (n = 2)* | 1.3 × 10⁷ | 0 | 0 | 0 |
| 25 (n = 1) | 1.3 × 10⁷ | 0 | 0 | 0 |
| 10 (n = 1) | 1.2 × 10⁷ | 0 | 0 | 0 |
| 5 (n = 1) | 9.0 × 10⁶ | 0 | 0 | 0 |
| 2 (n = 2) | 4.6 × 10⁶ | 2.2 × 10⁴ | 0 | 0 |
| MRSA | | | | |
| 100 (n = 5) | 6.2 × 10⁷ | 0 | 0 | 0 |
| 25 (n = 1) | 6.4 × 10⁷ | 0 | 0 | 0 |
| 10 (n = 1) | 1.4 × 10⁸ | 0 | 0 | 0 |
| 5 (n = 1) | 3.6 × 10⁷ | 0 | 0 | 0 |
| 2 (n = 2) | 2.2 × 10⁷ | 0 | 0 | 0 |

### EXAMPLE 7

Introduction: An important consideration in hospital hygiene is hand cleanliness. Purell™ (Gojo Industries Inc, USA), is an alcohol-based hand gel that is currently widely used by nursing staff in hospitals in the UK. The copper metallo-ion composition CuAL42 has been shown herein to have potent biocidal activity against 5 common pathogenic bacterial strains. Consequently, an alcohol-free hand gel based on Aloe vera and containing 314 ppm of CuAL42 called Xgel has been formulated and compared to Purell in this example. The protocol used was based on EN (European Norm) 12054 (1997), a standardized procedure where the product under test must produce a 4 Log kill in 60 seconds in order to achieve the required standard.

Abbreviations: ACCB, Acinetobacter sp.; BSA, bovine serum albumin; cfu, Colony forming units; MRSA, methicillin-resistant Staphylococcus aureus; PBS, phosphate-buffered saline;

Results: As shown in FIGS. 5 to 7, in the case of MRSA and ACCB respectively, both Purell™ and Xgel both achieved the required 4 Log kill in 60 seconds. However, in both cases Xgel was considerably more effective than Purell, in that Xgel killed 100% of both strains of bacteria. In the case of C. difficile spores, Purell was ineffective, whilst Xgel very nearly achieved (3000-fold kill) the required 4 Log kill in 60 seconds.

Materials and Methods: The standard EN 12054 (1997) protocol was followed. Briefly, 9 ml of the test hand gel was inoculated with 1 ml of bacterial suspension and mixed. One ml aliquots were then taken at 30 and 60 seconds and mixed with 9 ml of Ringer's solution for 5 min. An aliquot was then taken and spread onto an agar plate and incubated overnight when CFUs were counted.

Discussion: Hand cleanliness is of great importance in hospital hygiene since bacteria or their spores can easily be spread around hospitals by hand contact. Purell™ is an alcohol-based hand gel that is currently widely used by health workers in UK hospitals.

The results of the present studies clearly show that Xgel, an Aloe vera-based hand gel that contains 314 ppm CuAL42, is considerably more effective against 3 important pathogenic bacteria—MRSA, Acinetobacter sp. and C. difficile spores—than Purell™. In this respect, it is important to note that C. difficile has become a greater threat to patient health than MRSA and more patients are now dying from C. difficile infections than MRSA.

Purell™, like all alcohol-based hand gels, is known in repeated, prolonged use to cause skin dryness and cracking. In contrast, Xgel being alcohol-free and having an Aloe vera base is much kinder to hands. Furthermore, our preliminary studies indicate that the residue from Purell™ left behind when the alcohol has evaporated can still support growth of MRSA and Acinetobacter sp. for at least 3 hours, whilst Xgel residue does not permit the survival of bacteria at all.

### EXAMPLE 8

### Report on Time-Kill Curves (TK) for MRSA and Acinetobacter sp (ACCB) Against Copper Compositions Coded CuAL42, CuPC33 and CuWB50, their Component Binders and Copper Sulphate Solution

### Introduction

We have shown (see FIGS. 17 to 19) that low concentrations of these copper compositions (CuAL42, CuPC33 and CuWB50) at one ppm achieved a three to four log kill over a two hour period. We have performed a range of time kill experiments at the minimal bactericidal concentration (MBC) as determined by MIC/MBC tube methods using RPMI-1460 medium and also at 150 ppm (as has been used in an experimental environmental cleaning situation).

### MIC/MBC Determinations

The MIC/MBC for each compound, relevant binder and copper sulphate was determined by making final concentrations of each ranging from 100 ppm down to 1 ppm in RPMI-1460 medium (Sigma) and then seeded with an inoculum of 2×10⁵ bacteria per tube. All tubes were incubated overnight at 37° C. and the MIC taken as the first tube to reveal no growth reading from 1 ppm upwards). The MBC was determined by subculturing all tubes showing no growth to blood agar, incubating overnight at 37° C. and reading for any growth of surviving colonies. The MBC is taken as the first tube to show no growth on agar plates (reading from the lowest concentration upwards).

### Time Kill Curves

Time kill curves were performed using RPMI-1460 medium (Sigma).

MRSA was tested at 20 ppm and at 150 ppm of each composition, binder and copper sulphate. (ref FIGS. 1 & 2) ACCB was tested at 40 ppm and 150 ppm of each composition, binder and copper sulphate (ref FIGS. 3 & 4). A growth control for each experiment consisted of RPMI-1460 and the test organism only.

Each reaction tube consisted of 10 ml of RPMI-1460 containing the required concentration of composition, binder or copper sulphate and was seeded with 2×10⁶ organisms and immediately incubated at 37 C. Aliquots were taken at points 0, 15, 30, 60, 120, 360 and 960 minutes and viable counts performed in triplicate using quarter strength Ringer's solution as diluent and neuturalizer seeded onto blood agar incubated overnight at 37° C. Colonies were counted and the count of survivors expressed as colony forming units. Log of the colony counts were plotted against each time point to produce a TK curve for each organism at each concentration against each compound, binder and copper sulphate. A curve for the growth controls were plotted on each curve series for comparison of growth rate. The term binder is used colloquially herein to embrace the components present in the copper compositions apart from the copper compound itself.

### Results Summary

Results of MIC/MBC determinations for MRSA were 10/20 ppm.

Results of MIC/MBC determinations for ACCB were 20/40 ppm.

### Time Kill Curves

Against MRSA: At 20 ppm CuAL42 and CuWB50 achieved a 4 log kill in 6 hours and a 6 log kill at some time between 6 and 16 hours. The log kill for CuPC33 was 3 log and 6 log respectively. At 150 ppm CuAL42 and CuWB50 achieved a 6 log kill after 60 minutes, CuPC33 after 120 minutes. All binders and copper sulphate had some activity but the bacteria recovered.

Against ACCB: At 40 ppm all three compositions achieved a 4 log kill after 6 hours and a 6 log kill between 6 and 16 hours. At 150 ppm all three compositions achieved a 6 log kill after 60 minutes. All binders and copper sulphate had little initial activity but the bacteria recovered.

The attached FIGS. 1 to 4 show the growth curves for each combination registered for 0, 15, 30, 60, 120 and 360 minutes and finally after 960 minutes (26 hrs incubation).

### EXAMPLE 9

### Decontamination Efficacy of a Non-Alcoholic Hand Gel Containing Copper-Based Biocides

Hand decontamination by application of purpose-made hand gels is essential for infection control. Most hand gels currently contain isopropyl alcohol, which bestows biocidal and rapid drying properties to the gel. Alcohol is neither friendly to the hands nor the environment, and is absorbed into the bloodstream. We formulated four non-alcoholic aloe vera hand gels, three including one of three inorganic biocides (CuWB50, CuAL42, and CuPC33) containing in the region of 300 ppm such as 314 ppm effective copper, and investigated whether these could decontaminate the hands as effectively as a commercial preparation. 10⁶ CFU or MRSA, or E coli, were applied to the hands of volunteers, and palm/finger imprints taken immediately afterwards. One of the four hand gels was then rubbed on the hands, and subsequent imprints were taken at timed intervals. Unlike the Aloe vera control, no MRSA could be retrieved from either the CuAL42 or CuWB50-containing gels immediately after application, and at all times afterwards. MRSA could be retrieved from CuPC33-treated hands for 15 minutes. Unlike the control, E coli could not be retrieved at any time point from hands treated with CuAL42-containing gel; complete disappearance of the organism was only seen at later time points for the other two gels. We conclude that CuAL42-containing gel rapidly and effectively eradicates viable organisms from hands, and may offer a more personally and ecologically acceptable alternative to alcohol-containing gels. Results are shown in FIGS. 5, 6 and 7.

### EXAMPLE 10

### Safety of CuAL42, CuPC33 and CuWB50

### Studies on the Cytotoxic Effects on Live Human Cells in Tissue Culture

### Background, Aims and Objectives

Other examples herein have established that these compositions have marked antibacterial activity, unexpectedly suoerior to the individual components. The present example sets out to investigate whether the antibacterial and toxic properties CuWB50, CuPC33, and CuAL42 towards bacterial pathogens extends to mammalian (human) cells.

### Materials and Methods

The three copper-containing antimicrobial solutions—CuPC33, CuAL42 and CuWB50—were provided and each contained 30.43 g/L of copper ion. A control solution of copper sulphate was made to the same concentration in distilled water. Two human cell lines were used for this example: HT-29, an intestinal epithelial cell line, and U937, a monocytic lymphoma. Samples of the copper-containing antibiotic solutions or copper sulphate at various concentrations in the appropriate complete media were added to established cell cultures and the cells cultured for a further 24 or 48 hours. After examination by microscopy the cells were then fixed and stained to quantitatively determine cytotoxicity using a sulforhodamine (SRB) cytotoxicity assay, developed and validated at the National Cancer Institute.

The percent of cytotoxicity of CuPC33 (▪), CuAL42 (▴), CuWB50 (▾) and copper sulphate (♦) was assessed using HT-29 cells at 24 and 48 hour time points and in media containing 5% or 25% fetal calf serum (FCS). All test cultures were in triplicate. Results are shown in FIG. 8.

The percent cytotixicity of CuPC33 (▪), CuAL42 (▴), CuWB50 (▾) and copper sulphate (♦) was assessed using U937 cells at 24 and 48 hour time points and in media containing 5% or 25% fetal calf serum (FCS). All test cultures were in triplicate. Results are shown in FIG. 9.

### Results

Examination by microscope revealed no obvious toxic effects of the copper-metallo-ion containing antibacterial solutions or copper sulphate at concentrations of 1-100 ppm on either cell line with 5% or 25% FCS. However, at 1000 ppm the copper-containing antibiotic solutions and copper sulphate caused rounding up of HT-29 cells in medium with 25% FCS, while HT-29 cells in medium with 5% FCS showed clear signs of cell death (rounding up with granular cytoplasm and loss of refractivity). These effects were similar in 24 and 48 hour cultures. HT-29 grew equally well in medium with 5% or 25% FCS (see control optical density values in the legend of FIG. 8 and increasing the serum concentration results in some protection against the cytotoxic effect(s) of the copper-containing antibiotic solutions. U937 cells grew better in medium with 25% FCS than in medium with 5% FCS (see control optical densities in the legend of FIG. 9), but showed similar patterns of cytotoxicity with the copper-containing antibiotic solutions and copper sulphate as HT-29.

The SRB assay results confirm that there was no significant cytotoxicity to either HT-29 cells (FIG. 8) or U937 cells (FIG. 9) by any of the 3 copper-metallo-ion containing antibacterial solutions or by copper sulphate at concentrations up to 100 ppm. With 1000 ppm there was generally 80-100% cytotoxicity by all 3 copper-containing antibacterial solutions at both 24 and 48 hours of culture with both cell lines. The modest protective effect of increased serum concentration cannot be distinguished by the SRB assay and emphasizes the value of microscopic evaluation of the cells. Copper sulphate was considerably less toxic to both HT-29 and U937 cells in medium containing 25% FCS (FIGS. 8 and 9, panels C and D).

### Conclusions

The 3 copper-containing antibiotic solutions CuPC33, CuAL42 and CuWB50 and copper sulphate were not significantly cytotoxic to 2 different human cell lines at concentrations from 1-100 ppm. At a concentration of 1000 ppm all 3 copper-containing antibiotic solutions were very cytotoxic (80-100%) to both human cell lines and this effect was only modestly reduced by higher FCS levels in the media. At 1000 ppm copper sulphate was also very toxic to both cell lines although this was substantially reduced by increasing the serum concentration and could be visualized by the SRB assay.

The results suggest that a very large biological safety window of toxicity of all three copper compositions exists as concerns their effect on bacterial, rather than mammalian (human) cells. This conclusion is based on the clear antimicrobial effects of the compositions at concentration ranges of 1 to 100 ppm, at which concentrations no cytotoxicity towards human cell lines could be detected.

### EXAMPLE 11

### The ability of CuAL42, CuWB50, and CuPC33 to Reduce or Eliminate the Bacterial Bioburden Present on Contaminated Cleaning Cloths

### Background, Aims and Objectives

Bacteria are most often removed from surfaces using either proprietary wet loop-based technologies, or the more modern (and effective) microfibre-based cloths. Ultramicrobfibre-based cloths (UMF) are particularly effective at removing bacteria from hard surfaces. These cloths work optimally with water containing no detergents. After use in the hospital environment, such cloths represent a biohazard, as they contain millions if not billions of viable organisms, at least some of which are known to be responsible for hospital-acquired infection. Since these cloths work optimally when dampened with water, we investigated whether addition of CuWB50, CuAL42, and CuPC33 to the water reduced or eliminated the viability of those organisms picked up by the cloths.

### Materials and Methods

Laminated surfaces were inoculated with buffered saline containing appropriate concentrations of MRSA, Acinetobacter, or Clostridium difficile spores, spread with a sterile flat spreader over a 100 square cm area and allowed to dry. The area was contact plated to ensure satisfactory deposition of live, viable pathogenic organisms. The area was then cleaned with ultramicrofibre cloths (UMF) moistened to the recommended limit of wetness with the respective copper composition at a final concentration of 75 ppm. The area was then contact plated again to assess the removal of the inoculum by the UMF. The UMF was then bagged in a mini-grip bag and left at room temperature for 16 hours to simulate travel to the laundry. After 16 hours the UMF was placed into 100 ml phosphate buffer and agitated in the Stomacher (a device designed to release viable organisms from fabrics and foodstuffs) for 3 minutes at 250 rpm. Viable bacterial counts were performed on the eluent and 10 ml of eluent centrifuged at 3500 rpm for 10 minutes and the deposit cultured onto blood agar. The background count of the boards and the counts of PBS were tested for any environmental contamination. The results are presented in Table 16 below.

TABLE 16

| | Contact plates | | | | | |
|---|---|---|---|---|---|---|
| | (expressed as | | | | | |
| | number of | | | | | |
| | bacteria | | Stomacher | | | |
| | recovered) | | eluent from | Board | | Inoculum |
| | Pre- | Post- | UMF/Cu after | surface | PBS | used per |
| Compound/organism | Clean | clean | 16 hr @ RT | control* | control** | 100 Sq. cm |
| CuAL42 | | | | | | |
| MRSA | >500 | 0 | 6.6 × 10² | 0 | 0 | 2 × 10⁶ |
| ACCB | >500 | 0 | 0 | 0 | 0 | 2 × 10⁶ |
| CD spores | >500 | 0 | 0 | 0 | 0 | 3 × 10⁵ |
| CuPC33 | | | | | | |
| MRSA | >500 | 0 | 6.6 × 10² | 0 | 0 | 2 × 10⁶ |
| ACCB | >500 | 0 | 0 | 0 | 0 | 2 × 10⁶ |
| CD spores | >500 | 0 | 0 | 0 | 0 | 3 × 10⁵ |
| CuWB50 | | | | | | |
| MRSA | >500 | 0 | 3.3 × 10² | 0 | 0 | 2 × 10⁶ |
| ACCB | >500 | 0 | 0 | 0 | 0 | 2 × 10⁶ |
| CD spores | >500 | 0 | 0 | 0 | 0 | 3 × 10⁵ |
| Control UMF | | | | | | |
| MRSA | | | 2 × 10⁶ | | | |
| ACCB | | | 2 × 10⁶ | | | |
| CD spores | | | 3 × 10⁵ | | | |

### Conclusions

Contact plating showed a viable inoculum that was effectively removed by the UMF. Complete kill was achieved by all three copper compositions in the 16 hour time frame against Acinetobacter and C. difficile spores and a four log kill (99.99%) against MRSA. There were no recoverable bacteria from the centrifuged deposit of the eluent from the Acinetobacter or the C. difficile UMF-Cu cloths. This example suggests that all three copper compositions present at 75 parts per million, are highly effective biocidal agents when used in conjunction with cloth cleaning technology currently being assessed and implemented across the NHS. Whilst other biocides (such as quaternary ammonium compounds, halides, etc) are equally effective in this context, it is likely that the current drive towards their elimination for environmental reasons will create the need for safer alternatives. The data presented here supports the premise that these copper metallo-ion compositions may offer such an alternative.

### EXAMPLE 12

### Efficacy of Copper Antimicrobials CuAL42 and CuPC33 Against H. pylori

In this example standard NCCLS methods are used for testing, using strains NCTC CagA positive, NCTC CagA negative, and ACTC J5 (genome sequence known). The clinical isolates were UK1 metronidazole resistant and B1 clarithromycin resistant. A final inoculum of log 7 cfu/ml (colony forming units per millilitre) was used.

In the method, a standard kill-curve at concentrations of 0.5, 1.0, 5.0 and 12 ppm of each of these antimicrobial products was derived from sampling at 15, 30, 60 and 120 minutes.

The neuturaliser used was ¼ Ringer's lactate. As to quantification, decimal dilutions were prepared and 100 microlitres plated. The plates were incubated for 5 days at 37 deg C. in an atmosphere generated by CampyGen.

### Results:

As depicted in the accompanying drawing FIGS. 10 to 14, the CuAL42 was more active than CuPC33. CuAL42 at 5 ppm reduced the viable count by 5 to 6 logs over 120 minutes. CuAL42 at 12 ppm reduced the viable count by 5 to 6 logs in 30 minutes and resulted in no growth in 60 to 120 minutes. Neither the cagA status nor the resistance to metronidazole or clarithromycin appeared to have any effect upon the efficacy of the two copper metallo-ion compositions.

### EXAMPLE 13

### Anti-MRSA Activity of Hand Gel Residues

Methods: The hand gels were spread on laminate surface boards at 1 ml per 10 cm² and allowed to dry overnight at room temperature. 0.1 ml of an MRSA suspension in PBS (106 CFU/ml) was carefully spread onto each 10 cm² marked area (one square for each time point for each hand gel residue) and allowed to dry for 10 minutes. The squares were immediately contact-plated (t=0 hours) and then at various time points up to 24 hours. The contact plates were incubated for 24 hours and the colony forming units (CFUs) counted.

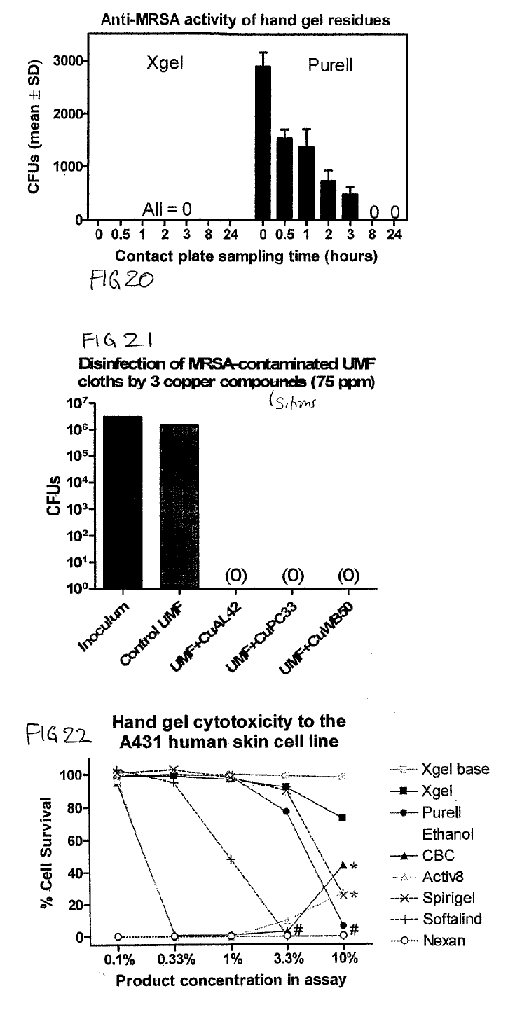

Results: As shown in FIG. 20, there were no CFUs at any time point on the Xgel residue, presumably owing to the presence of CuAL42 in the residue. In contrast, CFUs were detected at all time points up to 3 hours on the Purell residue, although these decreased in a time-dependent fashion, suggesting that a preservative or some other component in the residue has a modest antibacterial activity. This cannot be attributed to the presence of alcohol in the Purell residue as this would have evaporated during the overnight drying period.

Conclusion: The Xgel residue prevented survival and growth of MRSA at all time points, whilst the Purell residue supported MRSA survival for at least 3 hours. It is estimated by the NHS that 1 litre of Purell is used per bed per month. Since 1 litre of Purell contains 70% alcohol then around 300 ml of residue will be deposited around each bed per month and this can potentially support the survival of MRSA (and preliminary results showed similar results with an antibiotic-resistant Acinetobacter strain). In contrast, Xgel residue does not support the survival of MRSA (or Acinetobacter—preliminary results) and would therefore help to prevent bacterial growth and survival in healthcare settings.

### EXAMPLE 14

### Disinfection of MRSA-Contaminated UMF Cloths by Three Copper Compositions at 75 ppm

Methods: MRSA (2×10⁶) in PBS were spread on laminate surface boards (50 cm²) and allowed to dry for 10 minutes. One square was immediately wiped with an ultramicrofibre (UMF) cloth, stomached, plated and colony forming units (CFUs) were counted 24 hours later to confirm that the inoculum was correct and was fully taken up by the UMF cloth. The other boards were wiped with either a control UMF wetted with water or UMFs wetted with water containing 75 ppm of the 3 copper compositions. These contaminated UMFs were placed in plastic bags for 16 hours, then stomached, plated and CFUs were counted 24 hours later.

Results: As shown in FIG. 21, the inoculum control contained 2×10⁶ CFUs indicating that the UMF cloths take up all of the MRSA bacteria. The control UMF cloth wetted with only water and stored for 16 hours contained 1×10⁶ MRSA, whilst the UMF cloths wetted with the 3 copper compounds contained no live bacteria after storage for 16 hours.

Conclusion: These results clearly demonstrate that UMF cloths are very effective at removing MRSA from laminate surfaces, such as those used in hospitals. However, the survival of the bacteria on the UMF cloths is very good and disposal or washing of these cloths poses a serious risk of transmitting the live bacteria elsewhere. Therefore, the fact that the UMF cloths wetted with the copper compounds contained no surviving MRSA after 16 hours is very important. This 100% effective decontamination seen with the 3 copper compositions could be of great value in hospitals and other places where potentially dangerous bacteria need to be removed from surfaces.

### EXAMPLE 15

### Hand Gel Cytotoxicity to A431 Human Skin Cell Line

Methods: The human squamous epithelial cell line A431 was cultured in RPMI 1640 medium supplemented with 10% FCS, 2 g/L sodium bicarbonate and 2 mM L-glutamine (complete medium), in 75 cm² tissue culture flasks in a humidified incubator at 37° C. with a 5% CO₂ in air atmosphere. For the cytotoxicity experiments, A431 cells were plated into the wells of flat-bottom 96 well plates at 5×10⁴ cells per well in 200 μl of complete medium and allowed to grow to confluence. On the day of the experiment the depleted culture medium was aspirated and replaced with 100 μl of fresh complete medium. Samples of the hand gels were diluted in complete medium to double the concentrations shown in the Figure and 100 μl of each samples was added to the cells which were then cultured for a further 24 hours. After microscopic examination, the cells were fixed and stained to quantitatively determine cytotoxicity as described below. The sulforhodamine B (SRB) cytotoxicity assay was developed and validated at the National Cancer Institute. Briefly, the cells were washed twice with RPMI medium (no FCS) and then fixed with 10% trichloroacetic acid for 1 hour at 4° C. After washing twice with tap water the cells were stained with SRB (0.4% w/v SRB in 1% acetic acid) for 30 min at room temperature. After washing twice with tap water the remaining stain was dissolved in 10 mM Tris base and the optical density (O.D.) of the wells was measured on a Dynatech Multiplate ELISA reader at 540 nm. The percent cell survival was calculated by dividing the test O.D. by control O.D. and multiplying by 100.

Results: As shown in the accompanying FIG. 22, Xgel base (Aloe vera gel with xanthan gum and citric acid as thickeners) had no significant effect on A431 cell survival at any concentration tested. Xgel is a non-alcoholic hand gel which consists of Xgel base with 314 ppm of CuAL42, a copper-based biocide; this product reduced cell survival by around 25% at the highest concentration, but had no effect at lower concentrations. 10% ethanol reduced A431 cells survival by around 50% but had little effect at lower concentrations. Purell is an alcohol-based hand gel that is currently used in hospitals for hand disinfection. Purell contains 62% denatured alcohol plus isopropyl myristate, propylene glycol, tocopheryl acetate, ammonomethyl propanol, and it killed more than 95% of the A431 cells at a 10% concentration, but had little effect at lower concentrations. Spirigel and Softalind are also alcohol-containing hand gels, but whilst Spirigel had a profile similar to Purell, Softalind killed around 50% of the A431 cells at a concentration of just 1%. However, Softalind contains a mixture of denatured alcohol and propanol as well as PEG-6 caprylic/capric glycerides and diisopropyl adipate, which presumably accounts for its significantly more toxic effect on A431 cells. Nexan is a hand gel that contains 0.2% triclosan plus detergent and it was extremely cytotoxic, killing the A431 cells at all concentrations tested. At high concentrations (#) Nexan actually dissolved the A431 cells (microscopic observation), an effect most likely due to the detergent. Finally, the 2 cleaning products CBC and Activ8 which contain quaternary ammonium compounds were also very cytotoxic to A431 cells. At higher concentrations (*) these products stuck the dead A431 cells to the plastic plates (microscopic observation) giving the false impression that cell survival was improved.

Conclusions: The results show that the alcohol-containing hand gels have a modest cytotoxic effect to A431 skin epithelial cells in culture. However, these cytotoxic effects were seen at 1/10^{th} or less of the concentration at which these products are used on hands by healthcare staff, and it is well documented that Purell, for example, causes skin dryness and cracking with frequent daily use.

Xgel also exhibited very modest cytotoxicity at 1/10^{th} normal strength—approximately the same effect as Purell at 1/33^{rd} normal strength—an effect presumably due to the presence of the CuAL42 biocide since Xgel base had no significant effect on A431 cells at any concentration. These results suggest that Xgel would be kinder to skin than Purell; furthermore, other studies have shown that Xgel is considerably more effective at killing MRSA, antibiotic-resistant Acinetobacter and Clostridium difficile spores than Purell. In fact, Purell was completely ineffective against C. difficile spores and since this bacterium that can cause fatal diarrhoea is now a greater cause of death in hospitals than MRSA, the use of Xgel rather than Purell would appear to be a logical choice.

Nexan contains 0.2% triclosan and detergent and it killed A431 cells completely at all concentrations tested. Astonishingly, Nexan is used as a standard hand gel by healthcare staff in Italian hospitals. The 2 cleaning products CBC and Activ8, which contain quaternary ammonium compounds as their active ingredient, were also very cytotoxic to A431 cells, but since these products are presumably used by people wearing rubber gloves they would not cause skin problems.

### EXAMPLE 16

### Determination of the Susceptibility of Three Copper Compositions to Different Bacterial Species Isolated from Hospital Outbreaks

Aim: To determine the activity of three copper compositions on a range of bacteria, such as Enterobacteriaceae, Pseudomonads, Staphylococci and Enterococci.

### Summary

A total of 170 different bacterial isolates (22 Acinetobacter, 18 Enterobacter, 27 Klebsiella, 26 Enterococci, 10 Pseudomonas, 37 Serratia and 45 Staphylococci) were tested for susceptibility to three copper compositions using MIC determinations. Zone sizes varied from 11-31 mm showing no patterns of resistance.

### Materials

1) Copper compositions used, as defined herein and coded: CuAL42, CuWB50 and CuPC33 derived from embodiments 1 to 8 in table 1

2) Isosensitest agar (ISO Agar)

3) Isosensitest broth (ISO broth)

4) Antimicrobial Susceptibility Test Discs (OXOID CT0998B)

5) Sterile swabs obtained form stores

6) Overnight growth of bacterial cultures

### Method

Antimicrobial susceptibility test discs (OXOID CT0998B) were saturated with 20 ul of each of the copper compositions, dried separately in a hot air oven for two hours and stored at 4° C.

Bacterial cultures were inoculated onto to appropriate media (nutrient agar or MacConkey) and incubated overnight. 5 well-isolated colonies were touched with a loop and inoculated into 5 ml of Isosensitest broth. (ISO broth). The broths were incubated overnight aerobically at 36° C.-1+2O° C. The inoculum was prepared by vortexing the overnight broth and pipetting “x” drops of the overnight culture from a long plastic Pasteur pipette into 5 ml ISO broth as follows:

| | |
|---|---|
| Enterobacteriaceae | 1 drop |
| Pseudomonas | 1 drop |
| Enterococci | 5 drops |
| Staphylococci | 2 drops |

A sterile swab was dipped into the vortexed inoculum suspension, pressed against the wall of the tube and rotated to remove excess fluid. The plates were inoculated using a rotary plater. Using sterile forceps the discs were placed on the plate so that they were in complete contact with the agar. Once applied the disc was not removed.

### Reading

The zone of inhibition was measured where growth was inhibited by the composition.

Results were recorded.

A=CuAL42

B=CuWB50

C=CuPC33

Zone sizes given in mm

### Results.

Staphylococcus aureus

| | | A | B | C |
|---|---|---|---|---|
| EMRSA-15 | | | | |
| H040220409 | E15 B1 | 26 | 22 | 23 |
| H040220408 | E15 B3 | 27 | 22 | 22 |
| H040340351 | E15 B3 | 26 | 26 | 26 |
| H061500550 | E15 B5 | 27 | 25 | 27 |
| H061500522 | E15 B7 | 31 | 25 | 27 |
| H061440332 | E15 B1 | 30 | 27 | 27 |
| H061520148 | E15 B17 | 22 | 22 | 19 |
| H061520592 | E15 B8 | 24 | 25 | 25 |
| H061780511 | E15 B1 | 20 | 17 | 18 |
| H061780562 | E15 B2 | 30 | 27 | 25 |
| H061880414 | E15 B3 | 20 | 19 | 19 |
| H062040630 | E15 B3 | 24 | 20 | 21 |
| EMRSA-16 | | | | |
| H045180281 | E16A1 | 30 | 28 | 25 |
| H040220405 | E16 A16 | 25 | 22 | 21 |
| H053000200 | E16 A14 | 25 | 22 | 21 |
| H055140586 | E16A12 | 23 | 21 | 20 |
| H060620616 | E16 A16 | 24 | 22 | 20 |
| H060620609 | E16 A2 | 22 | 22 | 23 |
| H060780341 | E16 A11 | 22 | 22 | 19 |
| H061620087 | E16 A7 | 24 | 22 | 20 |
| H061700478 | E16 A29 | 27 | 22 | 19 |
| H060780344 | E16A1 | 21 | 21 | 21 |
| H060440423 | E16A14 | 23 | 22 | 20 |
| H060200417 | E16 A16 | 20 | 19 | 18 |
| EMRSA-1 | | | | |
| H043980582 | GOS | 26 | 26 | 26 |
| EMRSA-17 | | | | |
| H041940150 | S'hampton | 26 | 26 | 26 |
| H053100245 | S'hampton | 26 | 26 | 25 |
| Irish-1 | | | | |
| H042280049 | Belfast | 25 | 25 | 25 |
| H054360295 | Craigavon | 25 | 24 | 22 |
| Irish-2 | | | | |
| H052080391 | Craigavon | 27 | 26 | 24 |
| CA-MRSA | | | | |
| H043880199 | ST1 PVL− | 25 | 24 | 22 |
| H060180184 | ST5 PVL+ | 25 | 25 | 25 |
| H045260142 | ST8 PVL+ | 27 | 24 | 22 |
| H044300316 | ST22 PVL+ | 22 | 19 | 17 |
| H060640427 | ST30 PVL+ | 27 | 25 | 24 |
| H060660187 | ST59 PVL+ | 24 | 23 | 22 |
| H054960270 | ST80 PVL+ | 25 | 25 | 25 |
| H052320141 | ST88 PVL+ | 25 | 25 | 25 |
| MSSAs | | | | |
| 55/3488 | 80/81; PVL+ | 27 | 27 | 26 |
| H051680084 | Distinct | 26 | 25 | 22 |
| H051760098 | Group II | 24 | 24 | 23 |
| | MSSA | | | |
| H051660517 | Group II | 24 | 24 | 24 |
| | MSSA | | | |
| H051260160 | Group II | 27 | 24 | 25 |
| | MSSA | | | |
| H051640376 | WSS-96 | 27 | 27 | 26 |
| H052260557 | Dis PVL+ | 27 | 25 | 24 |
| H060940449 | NT PVL+ | 26 | 22 | 12 |

| | | | | |
|---|---|---|---|---|
| VRE | VSE | A | B | C |

Enterococcus faecium

| | | | | | |
|---|---|---|---|---|---|
| H062940352 | POS | NEG | 29 | 28 | 31 |
| H062940351 | POS | NEG | 25 | 22 | 21 |
| H062760230 | POS | NEG | 32 | 28 | 30 |
| H062920531 | POS | NEG | 27 | 26 | 25 |
| H062940372 | POS | NEG | 26 | 24 | 25 |
| H063000437 | NEG | POS | 27 | 27 | 28 |
| H063000438 | NEG | POS | 27 | 24 | 29 |
| H062740365 | | | 30 | 26 | 25 |
| H062980090 | | | 31 | 27 | 33 |
| H062940548 | | | 32 | 29 | 31 |
| H062940550 | | | 28 | 24 | 27 |
| H062940547 | | | 29 | 24 | 26 |
| H062940549 | | | 28 | 24 | 26 |
| H062940322 | | | 30 | 25 | 29 |
| H062980250 | | | 30 | 27 | 28 |

Enterococcus faecalis

| | | | | | |
|---|---|---|---|---|---|
| H0630004390 | NEG | POS | 27 | 27 | 28 |
| H062980583 | POS | NEG | 29 | 27 | 29 |
| H062380292 | NEG | POS | 24 | 23 | 26 |
| H062960351 | | | 30 | 27 | 30 |
| H062960251 | | | 30 | 28 | 32 |

Enterococcus gallinarum

| | | | |
|---|---|---|---|
| H062980247 | 29 | 31 | 29 |

| Enterobacter cloacae | A | B | C |
|---|---|---|---|
| H062680089 | 17 | 16 | 20 |
| H062760216 | 19 | 18 | 19 |
| H062820406 | 17 | 14 | 20 |
| H062880482 | 15 | 11 | 18 |
| H062920526 | 14 | 11 | 13 |
| H062920437 | 23 | 19 | 25 |
| Outbreak strains | | | |
| Queen Elizabeth Hospital | | | |
| Gateshead QUEE09EB-1 | | | |
| H050760267 | 19 | 16 | 18 |
| H043820094 | 16 | 15 | 19 |
| H043820095 | 17 | 17 | 20 |
| H043820096 | 18 | 17 | 19 |
| H050760271 | 17 | 16 | 20 |
| St Georges Hospital HEB5 | | | |
| H0961460503 | 16 | 15 | 15 |
| H061460504 | 16 | 15 | 15 |
| H042360326 | 14 | 14 | 13 |
| H042360328 | 13 | 13 | 17 |
| H042360329 | 16 | 15 | 16 |

| Klebsiella pneumoniae | A | B | C |
|---|---|---|---|
| Outbreak strains HKL83 Liverpool | | | |
| H061720323 | 19 | 16 | 25 |
| H061720324 | 17 | 17 | 24 |
| H061760360 | 18 | 17 | 22 |
| H061760361 | 17 | 20 | 22 |
| H061760362 | 18 | 18 | 17 |
| H061760363 | 17 | 16 | 20 |
| H061400267 | 18 | 17 | 23 |
| H062020317 | 18 | 17 | 24 |
| H061480383 | 18 | 17 | 17 |
| H061480364 | 18 | 18 | 18 |
| H061120437 | 17 | 15 | 22 |
| H061120438 | 16 | 15 | 23 |
| H061120439 | 15 | 16 | 22 |
| Routine strains | | | |
| H062840595 | 12 | 12 | 15 |
| H062840614 | 15 | 13 | 17 |
| H062840675 | 12 | 13 | 17 |
| H062860495 | 14 | 13 | 16 |
| H062880408 | 12 | 12 | 17 |
| H062880414 | 14 | 13 | 17 |
| H062880489 | 12 | 12 | 15 |
| H062900312 | 14 | 12 | 11 |
| H062920527 | 11 | 13 | 16 |
| H062920528 | 11 | 12 | 16 |
| H062920529 | 13 | 15 | 17 |
| H062920530 | 13 | 14 | 15 |
| H062920245 | 15 | 14 | 14 |
| H062920257 | 12 | 14 | 14 |

Pseudomonas aeruginosa Outbreak Strains HPA86 St Georges Hospital

| | A | B | C |
|---|---|---|---|
| H062880427 | 20 | 17 | 25 |
| H062880428 | 17 | 17 | 23 |
| H062680429 | 17 | 17 | 23 |
| H061820407 | 20 | 18 | 24 |
| H061420408 | 18 | 16 | 21 |
| H062500552 | 21 | 18 | 24 |
| H062500553 | 19 | 17 | 23 |
| H053940608 | 20 | 17 | 24 |
| H053940608 | 20 | 17 | 24 |
| H053940609 | 18 | 17 | 23 |

Serratia marcesens Outbreak Strains St, Mary's Neonatal Unit

| | A | B | C |
|---|---|---|---|
| H062880311 | 19 | 17 | 24 |
| H062880312 | 18 | 18 | 20 |
| H062880313 | 20 | 18 | 20 |
| H062880314 | 20 | 18 | 23 |
| H062880315 | 19 | 18 | 20 |
| H062880316 | 20 | 18 | 20 |
| H062880317 | 20 | 22 | 20 |

| Acinetobacter baumannii | | A | B | C |
|---|---|---|---|---|
| A/3009 | SE clone | 22 | 20 | 23 |
| H043260547 | SE clone | 22 | 20 | 22 |
| H061340585 | SE clone | 18 | 18 | 21 |
| A/3214 | OXA-23 clone | 20 | 16 | 22 |
| H044640092 | OXA-23 clone | 20 | 20 | 23 |
| H060800607 | OXA-23 clone | 19 | 18 | 20 |
| H044220140 | NW strain | 21 | 21 | 27 |
| H034940173 | Tstrain | 22 | 19 | 20 |
| H052600376 | Tstrain | 20 | 19 | 22 |
| H060560322 | Tstrain | 21 | 18 | 25 |
| 3/A/3311 | Sporadic 1 | 17 | 14 | 19 |
| H043860186 | Midlands 2 | 16 | 13 | 17 |
| H060980542 | Sporadic 3 | 20 | 17 | 16 |
| A/2875/1 | W strain | 11 | 11 | 13 |
| RUH2034 | W strain | 13 | 11 | 16 |
| H060800430 | BUAC-1 | 12 | 11 | 12 |
| H034560177 | OXA-23 clone 2 | 11 | 10 | 12 |
| H042220635 | OXA-23 clone 2 | 11 | 11 | 12 |
| H042900157 | Sporadic 2 | 12 | 11 | 13 |
| H041200198 | 24AC-1 | 22 | 20 | 23 |

### CONCLUSION

A total of 170 strains, 22 Acinetobacters, 18 Enterobacters, 27 Klebsiellas, 26 Enterococci, 10 Pseudomonas, 37 Serratias, and 45 Staphylococci were tested against the three copper compositions. There was no resistance. The zone sizes varied from 11-31 mm.

1. An antibacterial formulation which comprises:
(a) at least one water soluble copper compound able to form copper ions upon dissolution in an aqueous medium;
(b) at least one water soluble ammonium agent able to form ammonium ions upon dissolution in an aqueous medium;
(c) at least one water soluble acid, and
(d) an aqueous medium within which components (a), (b) and (c) are dissolved,
said formulation having (e) an acidic pH and (f) an electrolytic potential in excess of 50 millivolts.

2. A formulation as claimed in claim 1, wherein (a) comprises one or more inorganic copper salts, such as for example copper sulphate, copper chloride, copper nitrate.

3. A formulation as claimed in claim 1 or claim 2, wherein (b) comprises at least one inorganic ammonium salt or hydroxide.

4. A formulation as claimed in any preceding claim, wherein (c) comprises one or more inorganic acids, such as for example one of hydrochloric, sulphuric, nitric and phosphoric acids.

5. A formulation as claimed in any one of claims 1 to 3, wherein (c) comprises one or more acids selected from the group consisting of citric acid, malic acid, tartaric acid, acetic acid, lactic acid.

6. A formulation as claimed in any preceding claim, in which the aqueous medium comprises or essentially consists of pure distilled water.

7. A formulation as claimed in any preceding claim, in which the pH value (e) is less than 5, preferably less than 4, more preferably less than 3 most preferably less than 2.5.

8. A formulation as claimed in claim 7, in which the pH value (e) is 2 or less.

9. A formulation as claimed in any preceding claim, in which the value (f) of electrolytic potential is in excess of 100 millivolts, preferably in excess of 150 millivolts, more preferably in excess of 200 millivolts, even more preferably in excess of 300 millivolts such as in the range of 300 to 400 millivolts.

10. An antibacterial formulation as claimed in any preceding claim in which the aqueous medium comprises a gel base.

11. A formulation as claimed in claim 10, in which the gel base comprises Aloe vera and one or more thickeners.

12. A formulation as claimed in claim 11, in which the thickener comprises at least one xanthan gum.

13. A formulation as claimed in any one of claims 10 to 12, in which the copper compound (a) is an organic salt and present in a concentration of 25 to 500 ppm, preferably 50 to 400 ppm, more preferably 100 to 350 ppm.

14. A formulation as claimed in any preceding claim, which essentially consists of the stated components therein.

15. A formulation as claimed in claim 14 which consists of the stated components therein apart from the possible presence of any unavoidable impurities.

16. A formulation as claimed in any preceding claim, wherein the copper compound (a) is hydrated crystalline copper sulphate, and the acid (c) comprises one acid selected from the group consisting of: sulphuric acid, hydrochloric acid and phosphoric acid, and the ammonium agent (b) comprises one ammonium compound selected from the group consisting of: ammonium sulphate, ammonium chloride and ammonium phosphate.

17. And antibacterial formulation as claimed in any preceding claim for use in controlling the growth and/or reproduction of bacteria.

18. A formulation as claimed in claim 17, wherein the bacteria are difficult to treat or otherwise persistent bacteria.

19. A formulation as claimed in claim 18, in which the bacteria are nosocomial bacteria or otherwise drug-resistant bacteria.

20. A formulation as claimed in any preceding claim for use in the preparation of a medicament for use in treating bacteria or a bacterial infection.

21. A formulation as claimed in claim 20, in which the bacteria are difficult to treat or persistent bacteria such as nosocomial bacteria or otherwise drug-resistant bacteria.

22. Use of a formulation as claimed in any preceding claim as an antibacterial preparation.

23. A method of treating a surface or a material comprising bacteria, such as nosocomial or otherwise drug-resistant bacteria, which comprises applying to the said surface or material, a formulation as claimed in any one of claims 1 to 21.

24. An antibacterial formulation as claimed in any one of claims 1 to 21, in combination with at least one detergent.

25. A detergent composition which comprises one or more detergents in conjunction with an antibacterial formulation as claimed in any one of claims 1 to 21.

26. A material substrate which has been impregnated with at least one antibacterial formulation as claimed in any one of claims 1 to 21.

27. A substrate as claimed in claim 26, which is a tissue material.

28. A substrate as claimed in claim 26, which is textile or fabric material.

29. A substrate as claimed in claim 28, which is a cloth material.

30. A substrate as claimed in claim 29, which is a microfibre cloth material.

31. A substrate as claimed in claim 30, which is an ultra microfibre cloth material.

32. An antibacterial formulation which comprises a formulation as claimed in any one of claims 1 to 21 together with an acceptable carrier, diluent or excipient therefor.

33. A method of disinfecting a surface which comprises applying to the surface a material substrate as claimed in any one of claims 26 to 31.

34. A method laundering a material comprising bacteria, which comprises subjecting the material to washing using a formulation as claimed in claim 24 or a detergent composition as claimed in claim 25.

35. An antibacterial formulation as claimed in any one of claims 1 to 21, in the form of a crème, soap, wash, spray solution, dressing solution, irrigation solution or spray mist formulation.

36. A method of disinfecting a surface by subjecting the surface to a spray mist or fog of an antibacterial composition as defined in any one of claims 1 to 21 or 35.

37. A bacterial infection control system which involves (i) detection of bacteria, (ii) presentation of detected results, (iii) treatment of detected bacteria by surface application or spraying a composition as defined in any one of claims 1 to 21 or 35, (iv) repetition of detection step, and repetition of presentation step.

38. An infection control system as claimed in claim 37 in which detection step (i) is performed by micro fluidic assay.
